(19) United States
(12) Patent Application Publication
(10) Pub. No.: US 2025/0019345 A1
(43) Pub. Date: Jan. 16, 2025

(54) NEW CANNABINOID-GABAPENTINOID CONJUGATES AND USES THEREOF

(71) Applicant: LONDON PHARMACEUTICALS AND RESEARCH CORPORATION, London (CA)

(72) Inventor: Mahmoud Mohamed Abdrabo Moustafa, London (CA)

(21) Appl. No.: 18/709,123

(22) PCT Filed: Nov. 10, 2022
(86) PCT No.: PCT/CA2022/051659

§ 371 Date: May 10, 2024

Related U.S. Application Data
(60) Provisional application No. 63/277,745, filed on Nov. 10, 2021.

(51) Int. Cl. C07C 305/24; A61K 31/24; A61K 31/325; C07C 229/08; C07C 229/28; C07C 271/54

(52) U.S. Cl. CPC C07C 305/24; A61K 31/24; A61K 31/325; C07C 229/08; C07C 229/28; C07C 271/54

(57) ABSTRACT

Cannabinoid-gabapentinoid conjugates and their formulations possessing dual synergistic pharmacological effects to control neuropathic pain, multiple sclerosis, seizures and postherpetic neuralgia, restless leg syndrome, trigeminal neuralgia, fibromyalgia, diabetic neuropathy, anxiety and bipolar disorders, schizophrenia, sleep disorders, and other related pathological conditions. The conjugates improve the therapeutic potential for both component compounds, while reducing the addiction and substance abuse problems commonly observed with each component, when prescribed independently, thereby providing a solution for cannabinoid and gabapentinoid substance abuse disorders.

### FIELD OF THE INVENTION

The present invention relates to the field of medicinal chemistry. Specifically, it relates to

compounds and methods for treating human or animal subjects in need of treatment, comprising a cannabinoid covalently or ionically bonded with one or more gabapentinoids.

### BACKGROUND

Gabapentinoids are analogues of the neurotransmitter γ-aminobutyric acid (GABA) and are prescribed mainly for the treatment of epilepsy and neuropathic pain, in addition to many other off labels uses. Examples of gabapentinoids include: gabapentin, pregabalin, phenibut, tolgabide, progabide, picamilon, γ-amino-β-hydroxy buteric acid, cis-2-aminomethylcyclopropane carboxylic acid, (Z)-4-Amino-2-butenoic acid, Lesogaberan, γ-valerolactone, γ-hydroxyvaleric acid, γ-hydroxybutyric acid, γ-butyrolactone, baclofen, and gabamide.

They exert their pharmacological effect via modulating several signalling pathways in the central nervous system (CNS) including: Voltage-gated sodium channels, voltage-gated potassium channels, and voltage-sensitive Ca²⁺ channels containing the alpha-2 delta-1 subunit (Sills, Graeme J. Current opinion in pharmacology 6.1 (2006): 108-113.)

Pregabalin and gabapentin are the most clinically important members of the gabapentinoid family. Both molecules share a similar mechanism of action, inhibiting calcium influx and subsequent release of excitatory neurotransmitters; however, they possess different pharmacokinetic and pharmacodynamic profiles (Bockbrader, Howard N., et al. Clinical Pharmacokinetics 49.10 (2010): 661-669.). Rapid and linear absorption of pregabalin, in addition to a higher Cₘₐₓ value, provide pregabalin with some distinct pharmacokinetic advantages over gabapentin that translate into an improved pharmacodynamic effect (Bockbrader, Howard N., et al. Clinical Pharmacokinetics 49.10 (2010): 661-669.). In contrast, orally administered gabapentin exhibits slower, incomplete and saturable absorption—a nonlinear (zero-order) process—making its pharmacokinetics less predictable and variable. As a result, its clinical use is limited.

Clinical benefits of gabapentinoids, and in particular pregabalin and gabapentin, covers many areas. For instance, both are prescribed for the use in epilepsy (Panebianco, M., et al. Cochrane Database of Systematic Reviews 7 (2019).), different types of neuropathic pain (Attal, N1, et al. European Journal of Neurology 17.9 (2010): 1113-e88.), chronic postsurgical pain (Clarke, H., et al. Anesthesia & Analgesia 115.2 (2012): 428-442.), low back pain (Shanthanna, H., et al. PLoS medicine 14.8 (2017): e1002369.), prophylaxis of episodic migraine (Linde,

Mattias, et al. Cochrane Database of Systematic Reviews 6 (2013).), and anxiolytic disorders (Slee, A., et al. The Lancet 393.10173 (2019): 768-777). Both drugs have some benefits in other CNS disorders, either as mono- or combined therapy, such as bipolar disorder, alcohol craving, and opioid dependence (Berlin, R. K., et al. The Primary Care Companion for CNS Disorders 17.5 (2015) & Ahmed, S., et al. Frontiers in Psychiatry 10 (2019): 228.). In addition, some studies suggested the use of pregabalin and gabapentin in the management of alcohol withdrawal symptoms (Freynhagen, R., et al. CNS Drugs 30.12 (2016): 1191-1200 & Muncie Jr, H. L., et al. American Family Physician 88.9 (2013): 589-595.

Clinical applications of gabapentinoids are hampered by their serious side effect profiles. In this regards, common adverse reactions of pregabalin include some serious effects such as irritability, ataxia, and more importantly abuse and addiction (Driot, D., et al. British journal of clinical pharmacology 85.6 (2019): 1260-1269.). Pregabalin appeared to be more addictive than gabapentin regarding the magnitude of behavioral dependence symptoms, transitions from prescription to self-administration, and the durability of the self-administrations (Bonnet, U., and N. Scherbaum. European Neuropsychopharmacology 27.12 (2017): 1185-1215.)

On the other hand, cannabinoids are a heteromorphic group of compounds that modulate the endocannabinoid system with complex and attractive pharmacological actions. They can be classified into three main groups: a) endogenous or endocannabinoids e.g., arachidonoylethanolamide: b) natural or phytocannabinoids which are the active constituents of

Cannabis species e.g. delta-9-tetrahydrocannabinol (THC) and cannabidiol (CBD); and c) synthetic e.g. nabilone.

There is a growing body of evidence to suggest the clinical utility of cannabinoids in many conditions including chronic pain, inflammation, neurodegenerative disorders, epilepsy, addiction, insomnia, multiple sclerosis, cancer, obesity and anorexia (Whiting, P. F., et al. JAMA, 313, 2456-73).

Recently, cannabinoid and gabapentinoid combination therapy is gaining a lot of interest. For instance, coadministration of gabapentin with THC in chronic constriction injury model of neuropathic pain in C57BL6 mice, synergistically reduced allodynia, and more importantly increased the therapeutic window of THC (Atwal, Nicholas, et al. Neuropharmacology 144 (2019): 115-121). Arachidonyl-2′-chloroethylamide, a selective CB1 receptor agonist, is reported to reduce the acute adverse effects of pregabalin and its pharmacodynamic profile as well (Florek-Luszczki, M., et al. Fundamental & Clinical Pharmacology 29.4 (2015): 382-393.). The synergistic effect between cannabinoids and gabapentinoids, especially in the treatment of various types of pains, are supported by other studies. For instance, Łuszczki, J. J., and Magdalena F. reported notable benefits between a synthetic cannabinoid and pregabalin using mouse model of acute thermal pain (Łuszczki, J. J., and Magdalena F. Pharmacological Reports 64.3 (2012): 723-732). The synergistic effect between cannabinoids and GABA-related structure includes various CNS disorders such as seizures (Luszczki, J. J., et al. European journal of pharmacology 720.1-3 (2013): 247-254; Florek-Luszczki, M., et al. Pharmacology Biochemistry and Behavior 130 (2015): 53-58.).

Furthermore, the clinical benefits of cannabinoid-gabapentinoid combination therapy is also supported by clinical trials. Turcotte, D., et al. reported nabilone-gabapentin combination as a well tolerated therapy for Multiple Sclerosis-Induced Neuropathic Pain (Turcotte, D., et al. Pain Medicine 16.1 (2015): 149-159.). Another open-label comparison study documented some positive impact of using nabilone/gabapentin combination therapy in the management of neuropathic pain in patients with peripheral neuropathy (Bestard, J. A., Cory C. T. Pain Practice 11.4 (2011): 353-368.).

One aspect that hampers the utility of cannabinoid-gabapentinoid combination therapy is the substantial difference in their respective pharmacokinetic profiles. As a result, both components do not reach their targets at the same time or bioavailability, especially after oral administration. In particular, absorption of gabapentin and pregabalin, as examples of gabpentinoids, is mediated by L-amino transferase (LAT) transporters that also facilitate the absorption of neutral amino acids. As a result, gabapentinoids, in general, have good bioavailability that may exceed 90%, as in the case of pregabalin, with a Tₘₐₓ in the range of 1 hour. In contrast, tetrahydrocannabinol (THC) and cannabidiol (CBD), as examples of cannabinoids, have relatively low oral bioavailability (<20%), with a Tₘₐₓ in the range of 4 hours.

Accordingly, there is a need for new compounds, formulations, and methods to enhance the pharmacokinetic profile of, and reduce the addiction and substance abuse problems associated with, both gabapentinoids and cannabinoids, via enhancing the oral bioavailability and facilitating cellular penetration. Such compounds, formulations, and methods would permit the administration of lower doses and improve therapeutic outcomes significantly.

### SUMMARY OF THE INVENTION

A compound, according to the present invention, has the general formula I or pharmaceutically acceptable salts, hydrates, or solvates thereof, wherein:

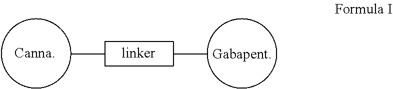

and wherein the cannabinoid is any chemical structure that modulates cannabinoid receptor(s), either as agonist, biased agonist, antagonist or with mixed action(s); the gabapentinoid is any compound that shares a structural similarity with GABA: and the linker is, a covalent or ionic bond, and it might be a chemical moiety with or without biological function(s). Each cannabinoid molecule might be linked with one or more gabapentinoid units, by way of the same or different linkers.

In another embodiment, chemical compounds of formula I have a cannabinoid chemical structure covalently or ionically linked with one or more gabapentinoid moieties using hydrolysable linkers. The present invention also relates to the medical applications of such compounds and their mono- or combined therapy with other therapeutics, and their preparations.

In another embodiment, the linker(s) may be covalent or ionic in nature. Covalent linkers may be linear, cyclic or branched alkyl carbon chains, functionalized or not, with different functionalities such as ester, amide, acetal, ketal, amino acid, short peptide, phosphate, phosphonate, each of which is optionally substituted. Ionic linkers include salts of carboxylates, phosphates, phosphonates, sulfates, sulfonates, sulfamates and related structures.

Cannabinoid and gabapentinoid moieties can be linked via a linker with additional pharmacological benefit(s) such as gallic acid that has antioxidant properties.

Certain exemplary compounds of the present invention include the following compounds of formulas 1-6, wherein R₁, R₂, and R₃ are selected from the tables below each formula.

| | |
|---|---|
| | 1 |
| 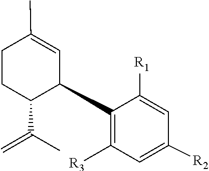 | |
| | 2 |
| 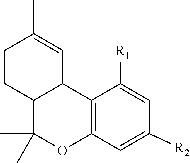 | |

| | R₁ | R₂ | R₃ |
|---|---|---|---|
| a. | 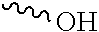 | 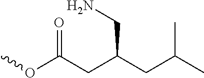 | 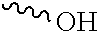 |
| b. | 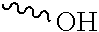 | 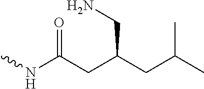 | 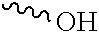 |
| c. | 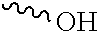 | 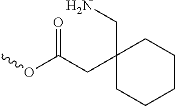 | 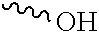 |
| d. | 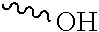 | 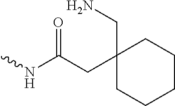 | 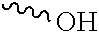 |
| e. | 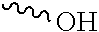 | 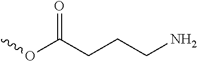 | 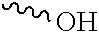 |
| f. | 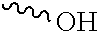 | 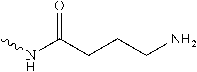 | 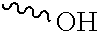 |
| g. | 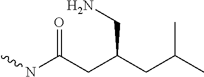 | 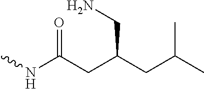 | 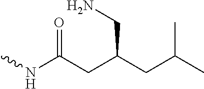 |
| h. | 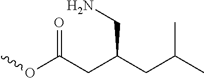 | 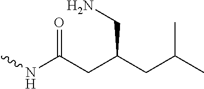 | 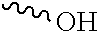 |
| i. | 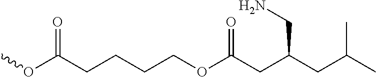 | heptyl | 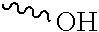 |
| j. | 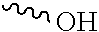 | 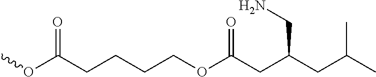 | 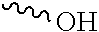 |
| k. | 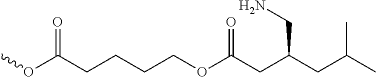 | pentyl | 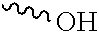 |
| l. | 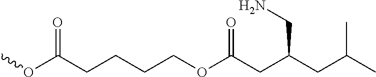 | propyl | 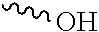 |
| m. | 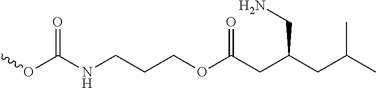 | heptyl | 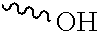 |
| n. | 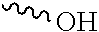 | 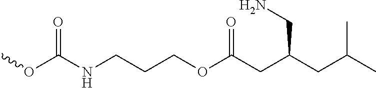 | 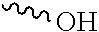 |
| o. | 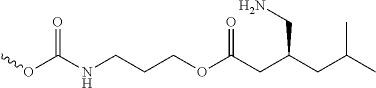 | pentyl | 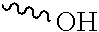 |
| p. | 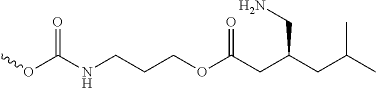 | propyl | 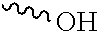 |
| q. | 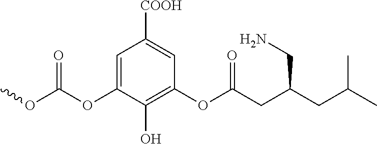 | heptyl | 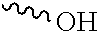 |
| r. | 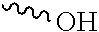 | 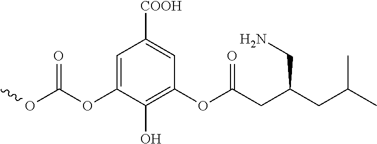 | 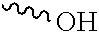 |
| s. | 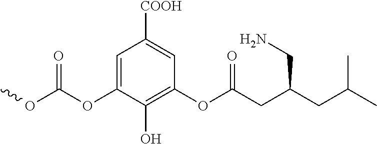 | 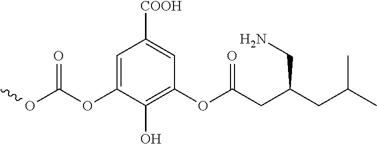 | 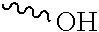 |
| t. | 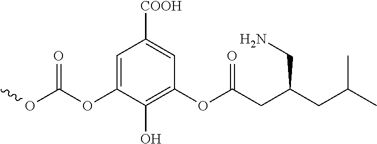 | 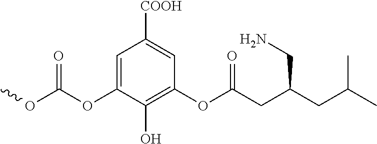 | 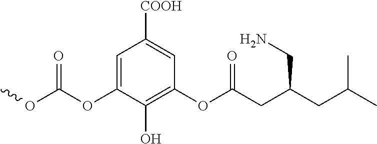 |
| u. | 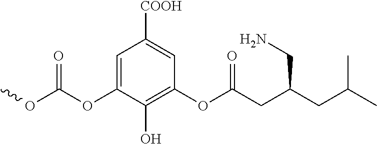 | pentyl | 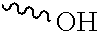 |
| v. | 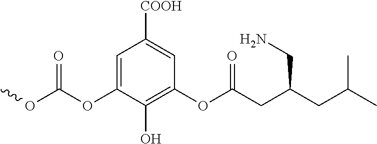 | propyl | 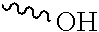 |
| w. | 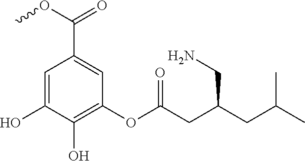 | heptyl | 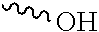 |
| x. | 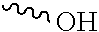 | 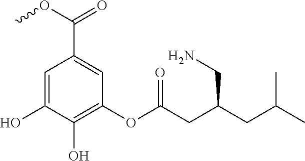 | 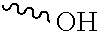 |
| y. | 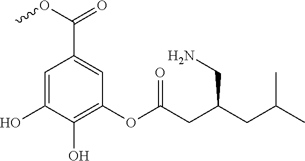 | 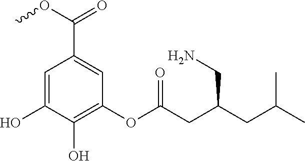 | 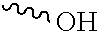 |
| z. | 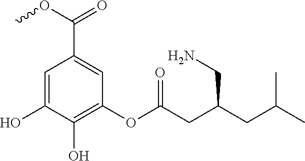 | 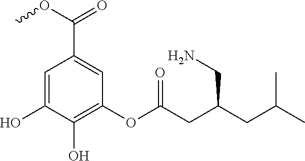 | 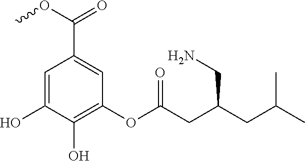 |
| aa. | 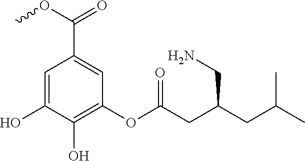 | pentyl | 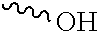 |
| bb. | 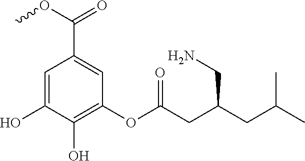 | propyl | 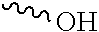 |
| cc. | 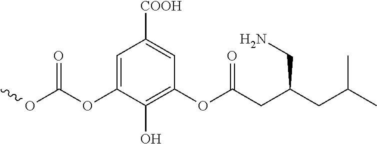 | heptyl | 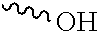 |
| dd. | 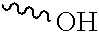 | 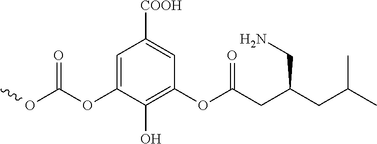 | 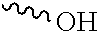 |
| ee. | 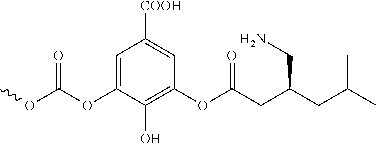 | 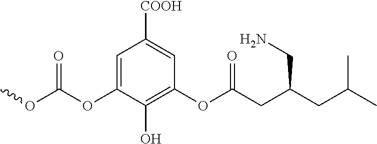 | 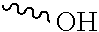 |
| ff. | 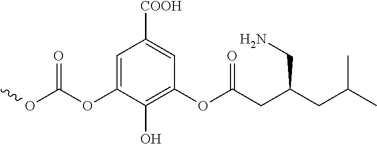 | 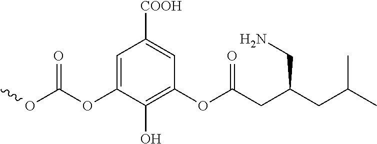 | 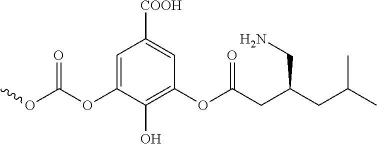 |
| gg. | 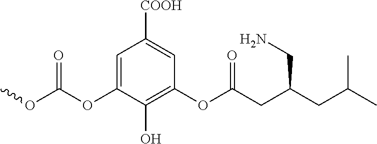 | pentyl | 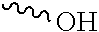 |
| hh. | 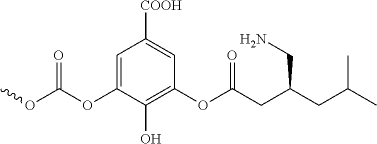 | propyl | 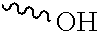 |
| ii. | 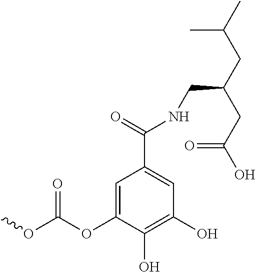 | heptyl | 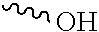 |
| jj. | 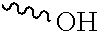 | 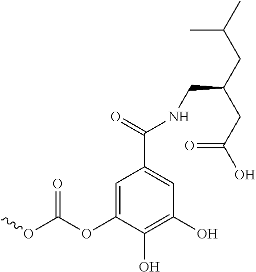 | 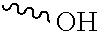 |
| kk. | 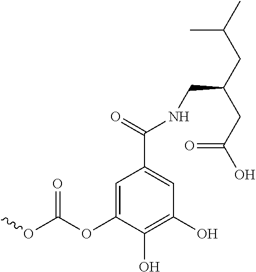 | 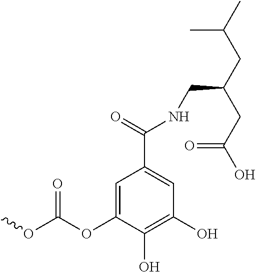 | 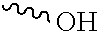 |
| ll. | 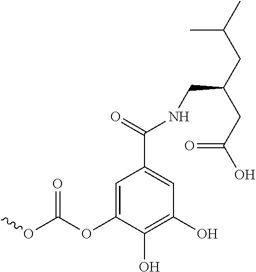 | 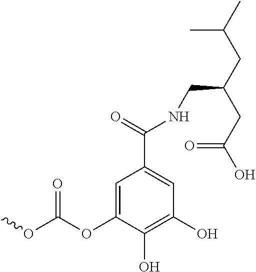 | 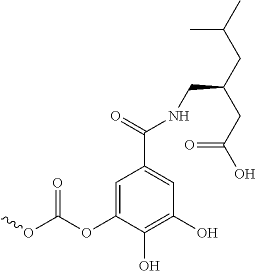 |
| mm. | 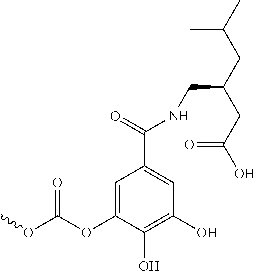 | pentyl | 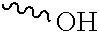 |
| nn. | 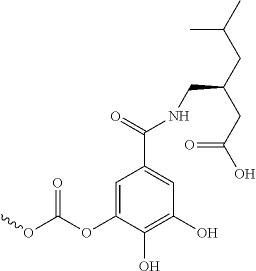 | propyl | 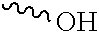 |

| | |
|---|---|
| | 3 |
| 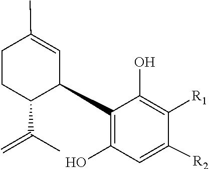 | |
| | 4 |
| 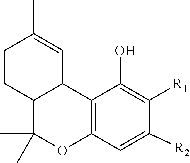 | |

| | R₁ | R₂ |
|---|---|---|
| a. | 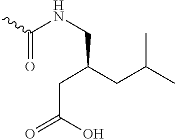 | pentyl |
| b. | 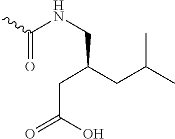 | propyl |
| c. | 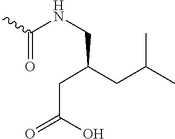 | 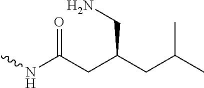 |
| d. | COOH | 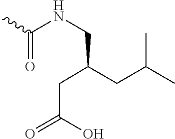 |
| e. | COOH | 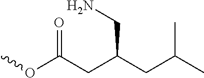 |
| f. | 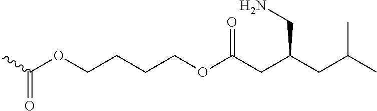 | pentyl |
| g. | 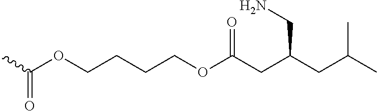 | propyl |
| h. | 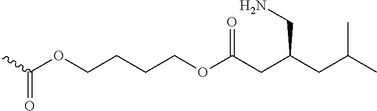 | 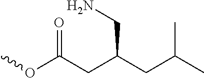 |
| i. | 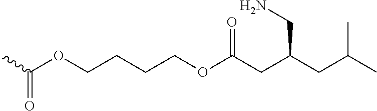 | 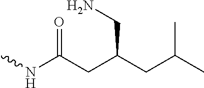 |
| j. | COOH | 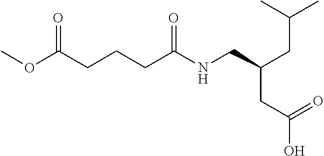 |
| k. | COOH | 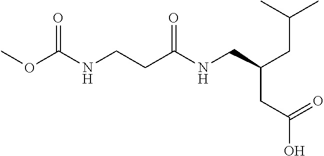 |
| l. | 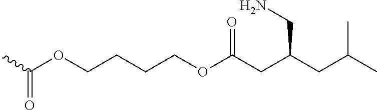 | 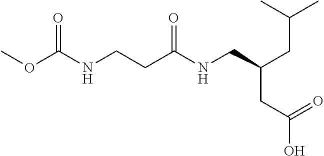 |
| m. | 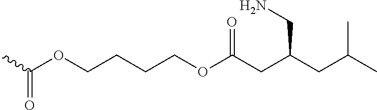 | 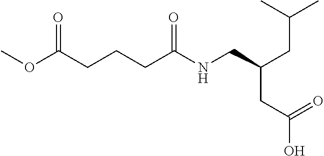 |
| n. | COOH | 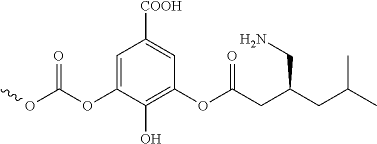 |
| o. | 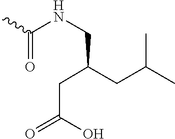 | 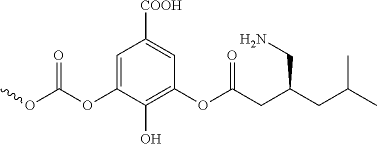 |
| p. | 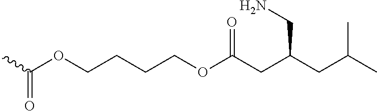 | 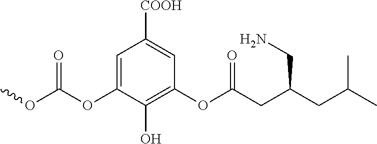 |
| q. | COOH | 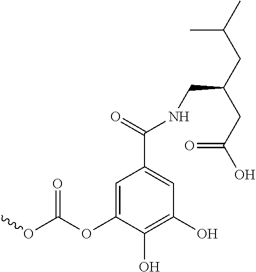 |
| r. | 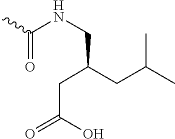 | 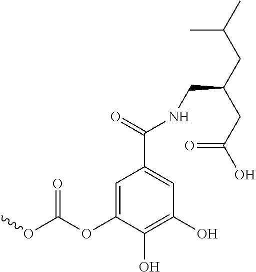 |
| s. | 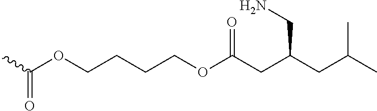 | 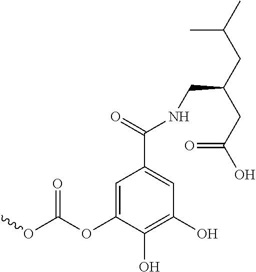 |

| | |
|---|---|
| | 5 |
| 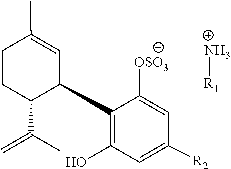 | |
| | 6 |
| 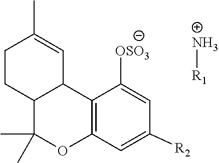 | |

| | R₁ | R₂ |
|---|---|---|
| a. | 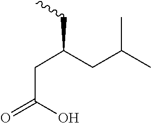 | pentyl |
| b. | 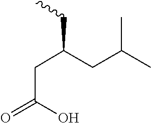 | propyl |
| c. | 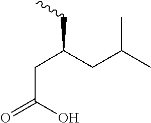 | 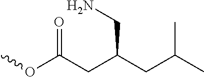 |
| d. | 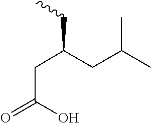 | 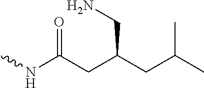 |
| e. | 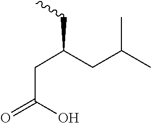 | 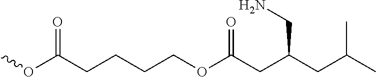 |
| f. | 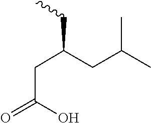 | 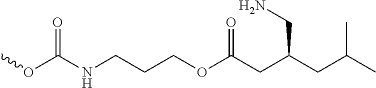 |
| g. | 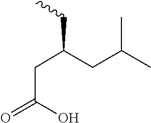 | 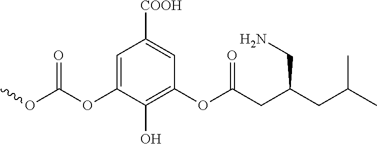 |
| h. | 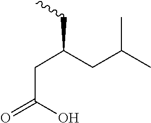 | 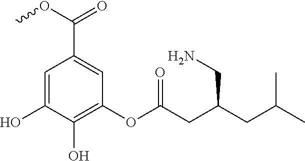 |
| i. | 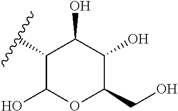 [SMILES: *[C@H]1C(O)O[C@H](CO)[C@@H](O)[C@@H]1O] | 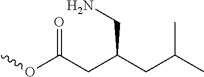 |
| j. | 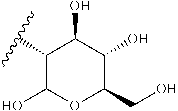 [SMILES: *[C@H]1C(O)O[C@H](CO)[C@@H](O)[C@@H]1O] | 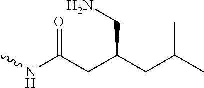 |
| k. | 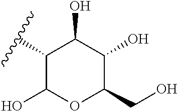 [SMILES: *[C@H]1C(O)O[C@H](CO)[C@@H](O)[C@@H]1O] | 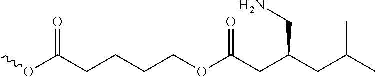 |
| l. | 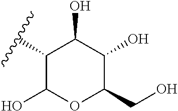 [SMILES: *[C@H]1C(O)O[C@H](CO)[C@@H](O)[C@@H]1O] | 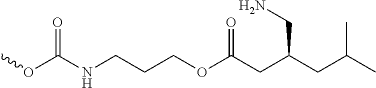 |
| m. | 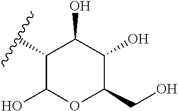 [SMILES: *[C@H]1C(O)O[C@H](CO)[C@@H](O)[C@@H]1O] | 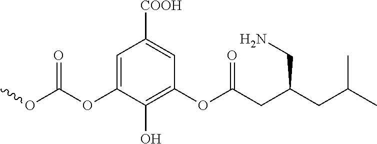 |
| n. | 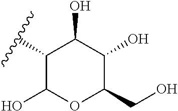 [SMILES: *[C@H]1C(O)O[C@H](CO)[C@@H](O)[C@@H]1O] | 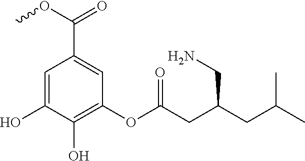 |

The cannabinoid-gabapentinoid conjugates. according to the present invention, unexpectedly modulate the cannabinoid receptors in a specific and potent mode of binding. Simultaneously. they are labile esters and salts of a cannabinoid derivative with a gabapentinoid that possesses a synergistic or additive effect. These new conjugates aim to deliver multiple synergistic or additive therapeutic benefits via more than one mechanism of action within the same time frame, mainly to manage pain and other CNS diseases. The conjugates also assist in overcoming the addiction and substance abuse problems associated with cannabinoids and gabapentinoids.

In another embodiment, the cannabinoid is one or more cannabinoids selected from the group consisting of: delta-9-tetrahydrocannabinol (THC), delta-8-tetrahydrocannabinol (THC), cannabidiol (CBD), cannabinol (CBN), cannabinolic acid (CBNA), cannabigerol (CBG), cannabigerol (CBG), cannabigerovarin (CBGV), cannabichromene (CBC), cannabicyclol (CBL), canabivarol (CBV), tetrahydrocannabivarin (THCV), cannabidivarin (CBDV), cannabichromevarin (CBCV), cannabigerol monoethyl ether (CBGM), cannabigerolic acid monoethyl ether (CBGAM), cannabidiolic acid (CBDA), cannabigerovarinic (CBGVA), cannabichromenic acid (CBCA), cannabichromenic acid (CBCA), cannabidiol monomethylether (CBDM), cannabidiol-C4 (CBD-C4), cannabidivarinic (CBDVA), cannabidiorcol (CBD-C1), delta-9-tetrahydrocannabinolic acid A (THCA-A), delta-9-tetrahydrocannabinolic acid B (THCA-B), delta-9-tetrahydrocannabinolic acid-C4 (THCA-C4), delta-8-tetrahydrocannabinolic acid (delta-8-THCA), delta-8-tetrahydrocannabinol (delta-8-THC), delta-9-tetrahydrocannabinol-C4 (THC-C4), delta-9-tetrahydrocannabiorcolic acid (THCA-C1), delta-9-tetrahydrocannabiorcol-C1 (THC-C1), tetrahydrocannabivarinic acid (THCVA), cannabicycolic acid (CBLA), cannbicyclol (CBL), cannabicyclovarin (CBLV), cannabielsoic acid A (CBEA-A), cannabielsoic acid B (CBEA-B), cannabielsoin (CBE), cannabivarin, cannabinol-C4 (CBN-C4), cannabinol methylether (CBNM), cannabiorcol (CBN-C1), cannabinol-C2 (CBN-C2), cannabinodiol (CBND), cannabinodivarin (CBVD), cannabitriol (CBT), cannabitriolvarin (CBTV), dehydrocannabifuran (DCBF), cannabifuran, cannabicitran (CBT), cannabiripsol (CBR), ‘11-hydroxytetrahydrocannabinol’ (11-OH-THC), ‘11-nor-9-carboxy-tetrahydrocannabinol’ (THC-COOH), and their derivatives, synthetic analogues, related chemical structures and salts, and mixtures and combinations thereof. The cannabinoid may also be a metabolite of any of the cannabinoids listed above. Preferably, the cannabinoid is CBD, THC, CBDA, THCA or THC-V.

In another embodiment, the gabapentinoid is one or more gabapentinoids selected from the group consisting of: GABA, gabapentin, pregabalin, Phenibut, tolgabide, progabide, picamilon, γ-amino-β-hydroxybuteric acid, cis-2-Aminomethylcyclopropane carboxylic acid, (Z)-4-Amino-2-butenoic acid, Lesogaberan, γ-valerolactone, γ-hydroxyvaleric acid, γ-hydroxybutyric acid, γ-butyrolactone, baclofen, and gabamide. The gabapentinoid may also be a metabolite of any of the gabapentinoids listed above. Preferably, the gabapentinoid is GABA, pregabalin or gabapentin.

In another embodiment, the linker is a covalent bond, a linear, cyclic, or branched alkyl carbon chain, functionalized or not, with ester, amide, acetal, ketal, amino acid, short peptide, phosphate, phosphonate, each of which is optionally substituted. The linker may also be an ionic bond by way of a sulfate, phosphate, or related functional groups. Where there are multiple linkers within the same conjugate, they may be identical or not.

In another embedment, the linker is selected to release the cannabinoid and gabapentinoid components in the body of the subject in need when subjected to metabolic enzyme(s), or chemical hydrolysis.

In some embodiments, the original conjugates (prior to hydrolysis) unexpectedly modulate the cannabinoid receptors via a specific and potent mode of binding. Upon hydrolysis, the conjugates release pharmacologically active cannabinoids and gabapentinoids with an improved pharmacokinetic and pharmacodynamic profile, compared to coadministration.

In some embodiments, the conjugates improve the therapeutic utility for both cannabinoids and gabapentinoids, while reducing the addiction and substance abuse related problems commonly observed with cannabinoids and gabapentinoids when prescribed independently. As a result, the conjugates provide a novel solution for cannabinoid and gabapentinoid substance abuse disorders.

It will be appreciated that the compounds described herein may be used alone or in combination with other compounds that may be therapeutically effective by the same or different modes of action. In addition, it will be appreciated that the compounds described herein may be used in combination with other compounds that are administered to treat other symptoms of psychiatric disorders, such as compounds administered to relieve pain, nausea, vomiting, and the like.

### BRIEF DESCRIPTION OF THE DRAWINGS

In order that the invention may be more clearly understood, a preferred embodiment thereof will now be described in detail by way of example, with reference to the accompanying drawings, in which:

FIG. 1 is a molecular diagram, showing the calculated binding poses of CBD and an exemplary cannabinoid-gabapentinoid conjugate, according to the present invention, within the pocket of the CBI receptor.

### DESCRIPTION OF THE PREFERRED EMBODIMENTS

The cannabinoid-gabapentinoid conjugates or any of their derivatives or metabolites, according to the present invention, have a cannabinoid and a gabapentinoid connected directly, or through one or more linkers, by hydrolysable covalent or ionic bonds. Certain preferred compounds of the present invention, unexpectedly modulate the cannabinoid receptors via a specific and potent mode of binding of the original conjugates with the binding pocket of the cannabinoid receptor (before being broken down by hydrolysis into separate cannabinoid and gabapentinoid components). Upon hydrolysis, the conjugates release pharmacologically active cannabinoids and gabapentinoids with an improved pharmacokinetic and pharmacodynamic profile, compared to coadministration of cannabinoids and gabapentinoids.

In some embodiments, the conjugates improve the therapeutic utility for both cannabinoids and gabapentinoids, while reducing the addiction and substance abuse related problems commonly observed with cannabinoids and gabapentinoids when prescribed independently. As a result, the conjugates may also provide a novel solution for cannabinoid and gabapentinoid substance abuse disorders, in addition to other advantages of certain embodiments described herein.

In some illustrative embodiments, this invention pertains to compounds having formula I, or pharmaceutically acceptable salts, hydrate, or solvates thereof:

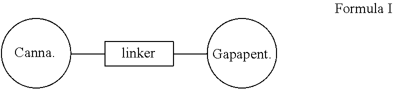

wherein the cannabinoid is any chemical structure that modulates cannabinoid receptor(s), either as an agonist, biased agonist, antagonist or with mixed action(s) and the gabapentinoid is any compound that shares a structural similarity with GABA. Specifically, the gabapentinoid is a compound with a carboxylic acid functionality or its isostere or analogues connected through a three-carbon chain to an amino group or its isostere or analogous. The linker is a covalent or ionic bond, and it may be a chemical moiety with or without biological function. Each cannabinoid molecule may be linked with one or more gabapentinoid units, with the same or a different linker.

Preferably, the gabapentinoid is a compound having formula II:

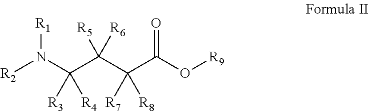

Where R₁ and R₂ are independently H, an alkyl group, a substituted alkyl group, an aceyl group, or a substituted aceyl group. Where R₃ to R₈ are independently H, a halogen, a nitrile group, hydroxide, an alkyl group, a substituted alkyl group, a cyclic alkyl group, a substituted cyclic alkyl group, an alkoxy group, and alkenyl group, a substituted alkenyl group, a cyclic alkenyl group, or a substituted cyclic alkenyl group. Where R₉ is H or an alkyl group. Each of the R groups between R₁ to R₉ may be part of a cyclic structure. Preferably, the gabapentinoid is GABA, pregabalin or gabapentin.

In certain specific examples this invention pertains to compounds of formula 1 or 2, or pharmaceutically acceptable salts, hydrate, or solvates thereof, wherein the R₁, R₂, and R₃ groups have formulas a to nn. The preferred conjugates are 1a-e and 2a-e.

| | |
|---|---|
| | 1 |
| 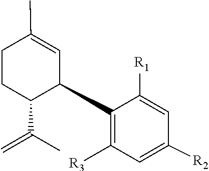 | |
| | 2 |
| 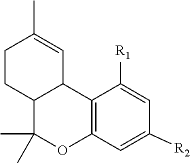 | |

| | R₁ | R₂ | R₃ |
|---|---|---|---|
| a. | 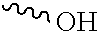 | 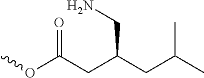 | 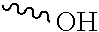 |
| b. | 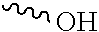 | 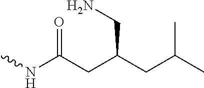 | 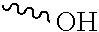 |
| c. | 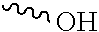 | 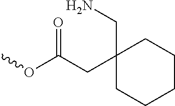 | 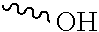 |
| d. | 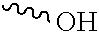 | 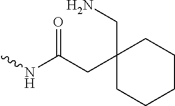 | 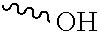 |
| e. | 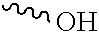 | 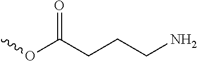 | 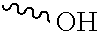 |
| f. | 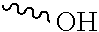 | 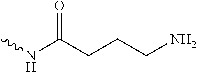 | 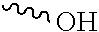 |
| g. | 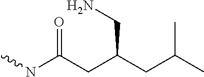 | 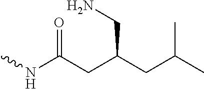 | 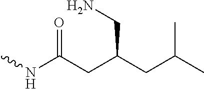 |
| h. | 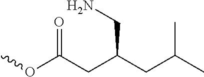 | 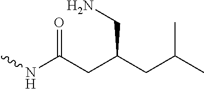 | 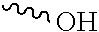 |
| i. | 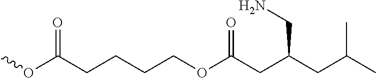 | heptyl | 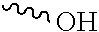 |
| j. | 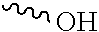 | 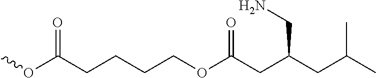 | 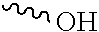 |
| k. | 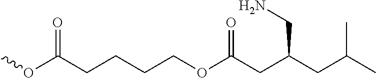 | pentyl | 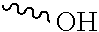 |
| l. | 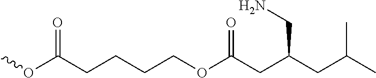 | propyl | 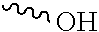 |
| m. | 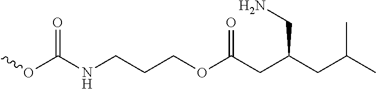 | heptyl | 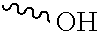 |
| n. | 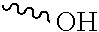 | 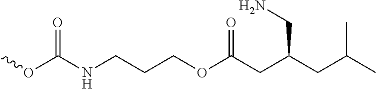 | 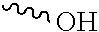 |
| o. | 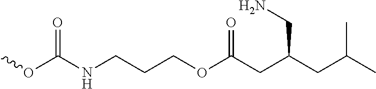 | pentyl | 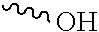 |
| p. | 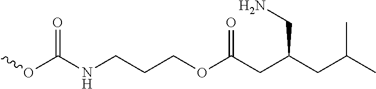 | propyl | 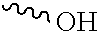 |
| q. | 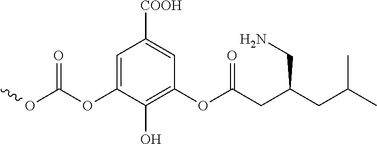 | heptyl | 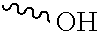 |
| r. | 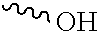 | 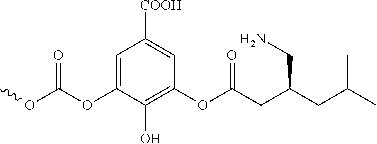 | 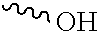 |
| s. | 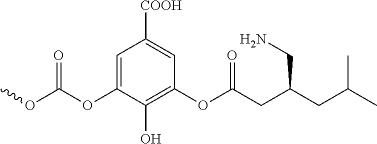 | 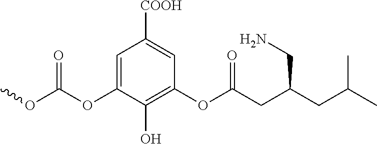 | 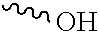 |
| t. | 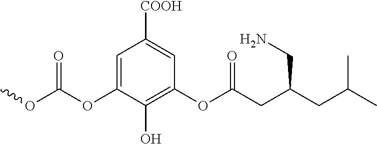 | 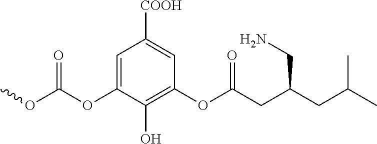 | 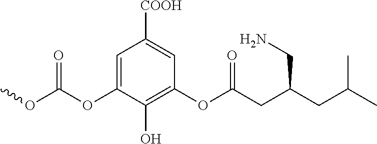 |
| u. | 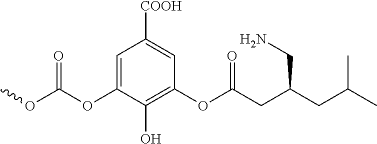 | pentyl | 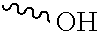 |
| v. | 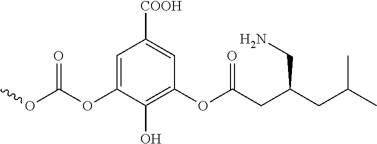 | propyl | 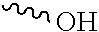 |
| w. | 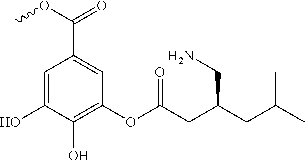 | heptyl | 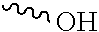 |
| x. | 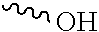 | 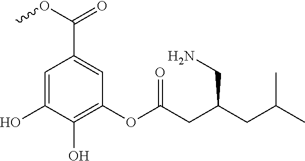 | 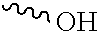 |
| y. | 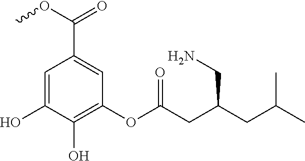 | 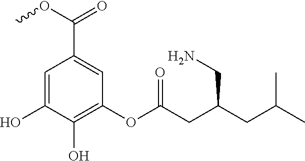 | 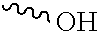 |
| z. | 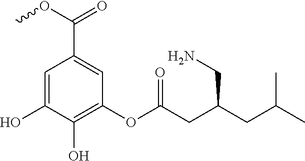 | 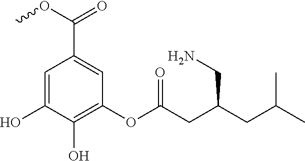 | 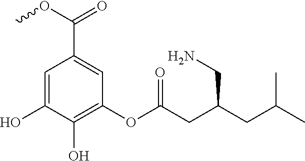 |
| aa. | 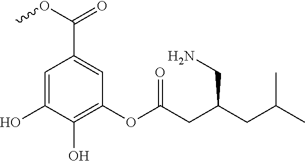 | pentyl | 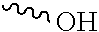 |
| bb. | 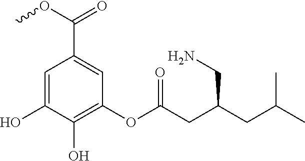 | propyl | 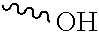 |
| cc. | 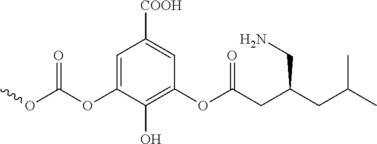 | heptyl | 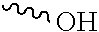 |
| dd. | 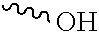 | 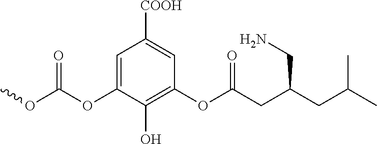 | 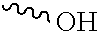 |
| ee. | 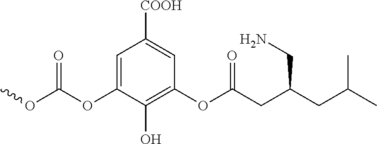 | 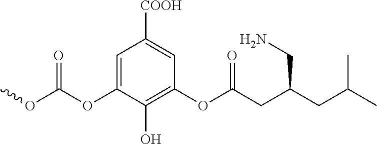 | 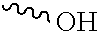 |
| ff. | 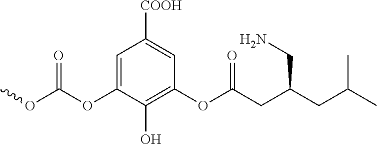 | 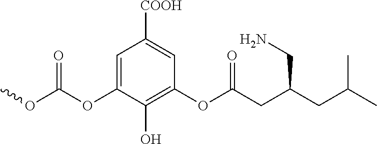 | 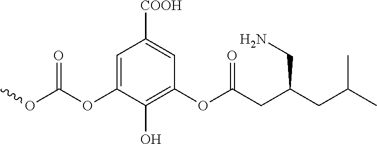 |
| gg. | 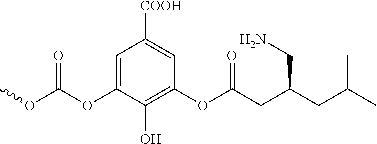 | pentyl | 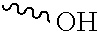 |
| hh. | 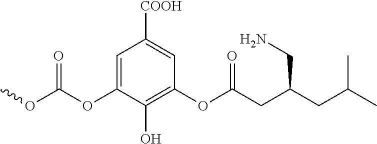 | propyl | 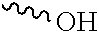 |
| ii. | 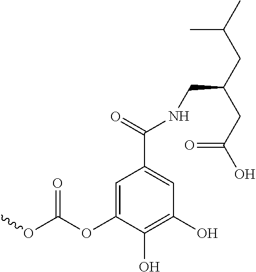 | heptyl | 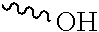 |
| jj. | 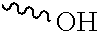 | 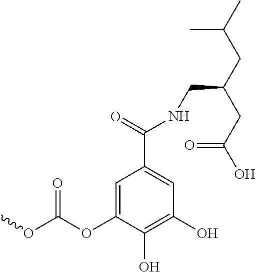 | 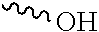 |
| kk. | 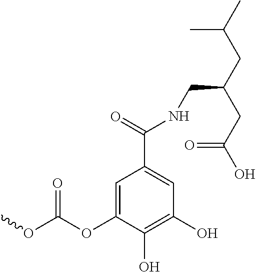 | 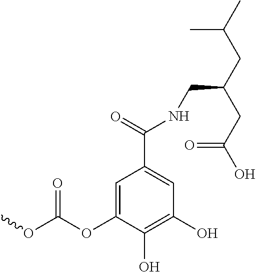 | 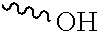 |
| ll. | 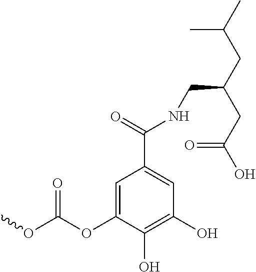 | 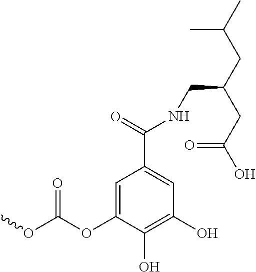 | 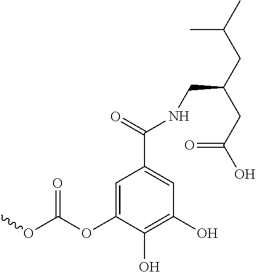 |
| mm. | 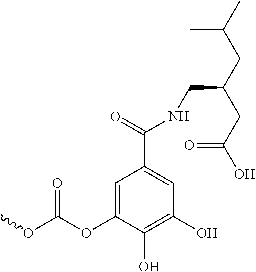 | pentyl | 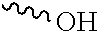 |
| nn. | 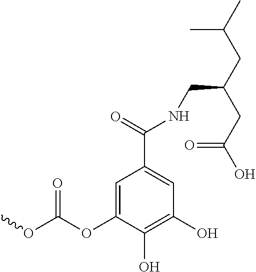 | propyl | 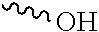 |

In further specific examples. this invention pertains to compounds of formula 3 or 4, or pharmaceutically acceptable salts, hydrate, or solvates thereof, wherein the R₁ and R₂ groups have formulas a to s. The preferred conjugates are 3a-b and 4a-b.

| | |
|---|---|
| | 3 |
| 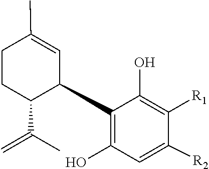 | |
| | 4 |
| 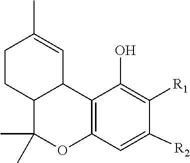 | |

| | R₁ | R₂ |
|---|---|---|
| a. | 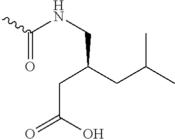 | pentyl |
| b. | 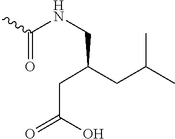 | propyl |
| c. | 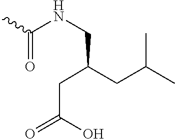 | 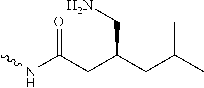 |
| d. | COOH | 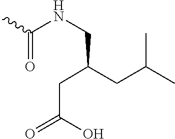 |
| e. | COOH | 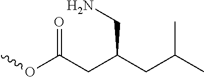 |
| f. | 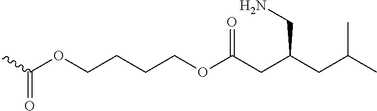 | pentyl |
| g. | 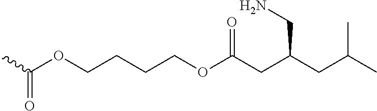 | propyl |
| h. | 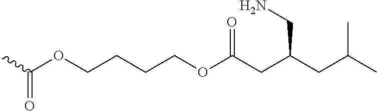 | 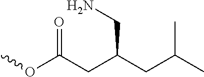 |
| i. | 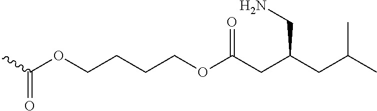 | 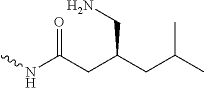 |
| j. | COOH | 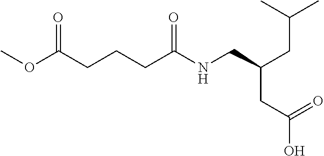 |
| k. | COOH | 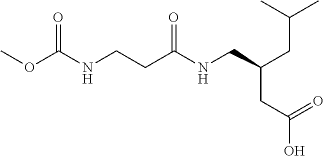 |
| l. | 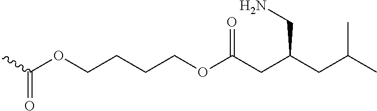 | 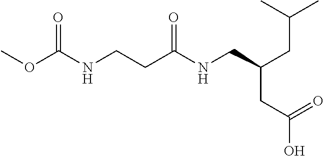 |
| m. | 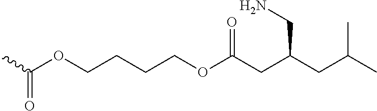 | 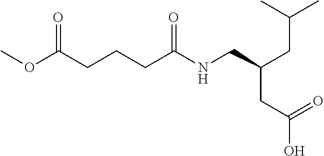 |
| n. | COOH | 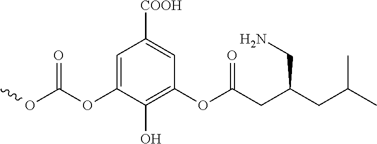 |
| o. | 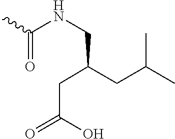 | 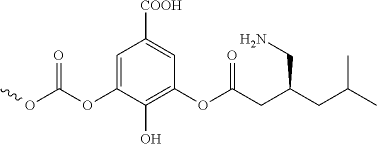 |
| p. | 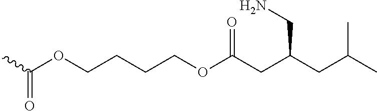 | 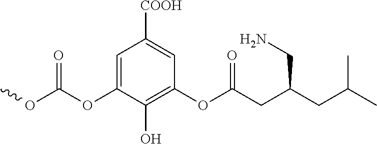 |
| q. | COOH | 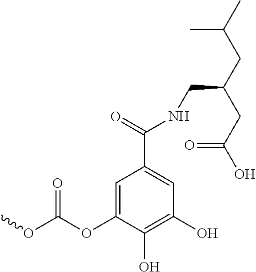 |
| r. | 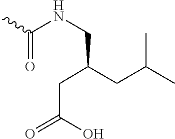 | 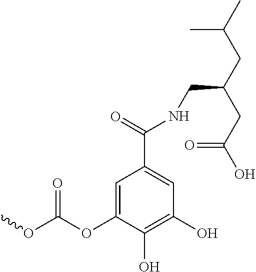 |
| s. | 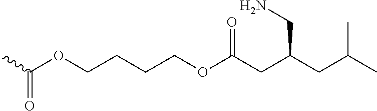 | 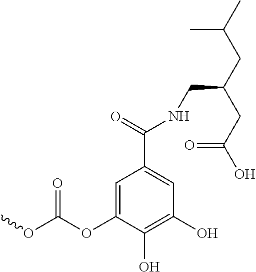 |

In further specific examples, this invention pertains to compounds of formula 5 or 6, or pharmaceutically acceptable salts, hydrate, or solvates thereof, wherein the R₁ and R₂ groups have formulas a to n. The preferred conjugates are 5a-b and 6a-b.

| | |
|---|---|
| | 5 |
| 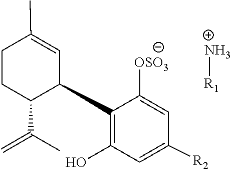 | |
| | 6 |
| 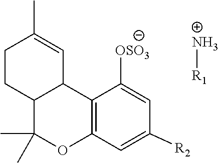 | |

| | R₁ | R₂ |
|---|---|---|
| a. | 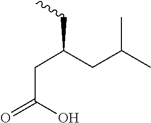 | pentyl |
| b. | 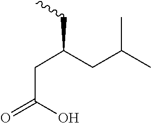 | propyl |
| c. | 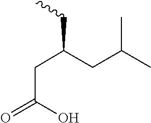 | 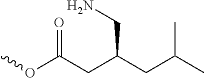 |
| d. | 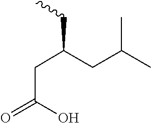 | 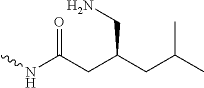 |
| e. | 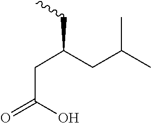 | 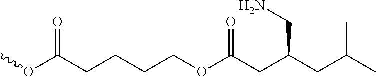 |
| f. | 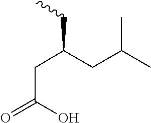 | 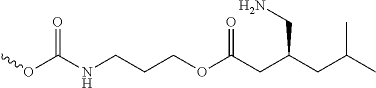 |
| g. | 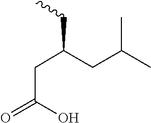 | 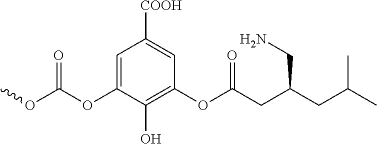 |
| h. | 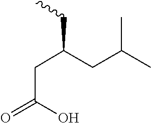 | 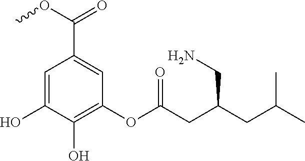 |
| i. | 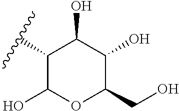 [SMILES: *[C@H]1C(O)O[C@H](CO)[C@@H](O)[C@@H]1O] | 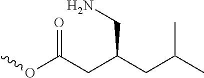 |
| j. | 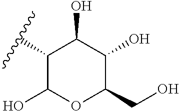 [SMILES: *[C@H]1C(O)O[C@H](CO)[C@@H](O)[C@@H]1O] | 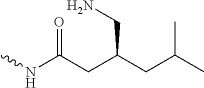 |
| k. | 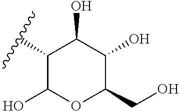 [SMILES: *[C@H]1C(O)O[C@H](CO)[C@@H](O)[C@@H]1O] | 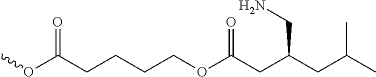 |
| l. | 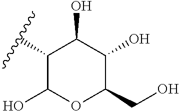 [SMILES: *[C@H]1C(O)O[C@H](CO)[C@@H](O)[C@@H]1O] | 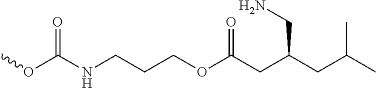 |
| m. | 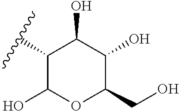 [SMILES: *[C@H]1C(O)O[C@H](CO)[C@@H](O)[C@@H]1O] | 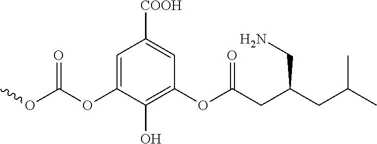 |
| n. | 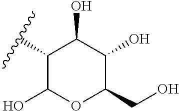 [SMILES: *[C@H]1C(O)O[C@H](CO)[C@@H](O)[C@@H]1O] | 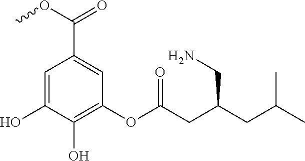 |

Conjugates with formula I are expected to release their active components after administration to a subject, as illustrated in scheme 1:

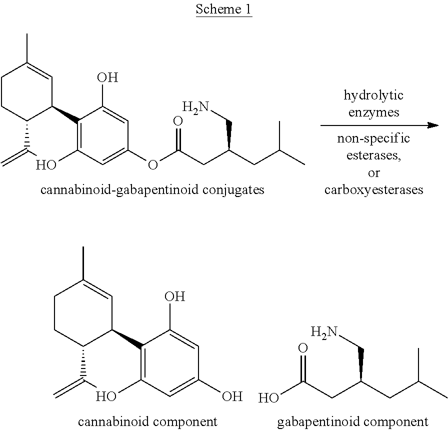 [SMILES: C=C1Oc2cc(OC(=O)C[C@@H](CN)CC(C)C)cc(O)c2[C@@H]2C=C(C)CC[C@@H]12 ; C=C1Oc2cc(O)cc(O)c2[C@@H]2C=C(C)CC[C@@H]12 ; CC(C)C[C@H](CN)CC(=O)O]

It is reported that a combined use of gabapentin and pregabalin produces a synergistic effect in pain control (Senderovich, H., Geetha, J. Current medical research and opinion 34.4 (2018): 677-682.). Therefore, conjugates of formula I may have a plurality of non-identical gabapentinoid components linked with the cannabinoid components, in order to produce a synergistic effect on the GABA system in the subject.

In certain specific examples, this invention pertains to compounds with formulas 7-11, in which the gabapentinoid component are non-identical, or pharmaceutically acceptable salts, hydrates, or solvates thereof:

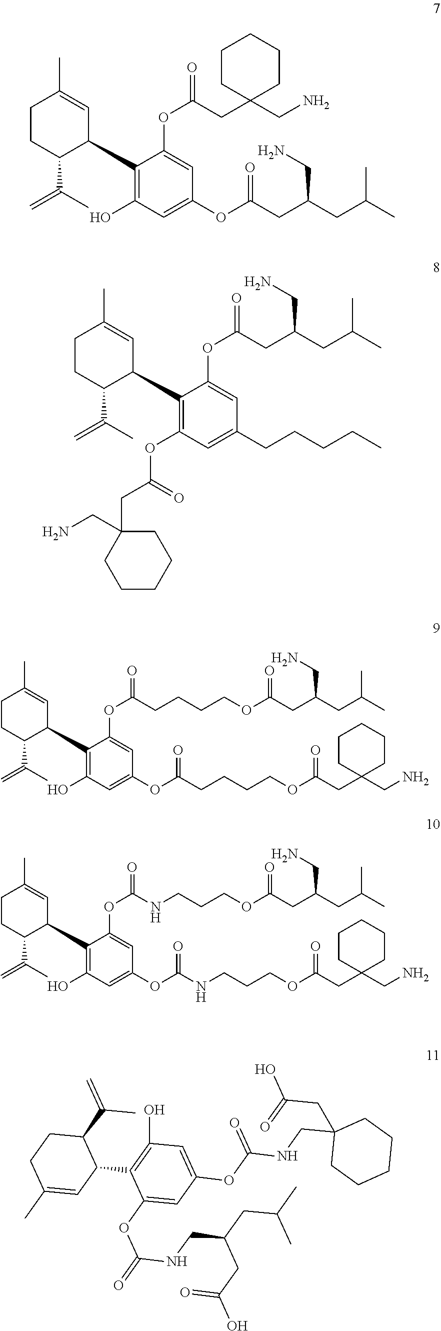

Conjugates of formula I with non-identical gabapentinoid components are expected to release their active components after administration to a subject, as illustrated in scheme 2:

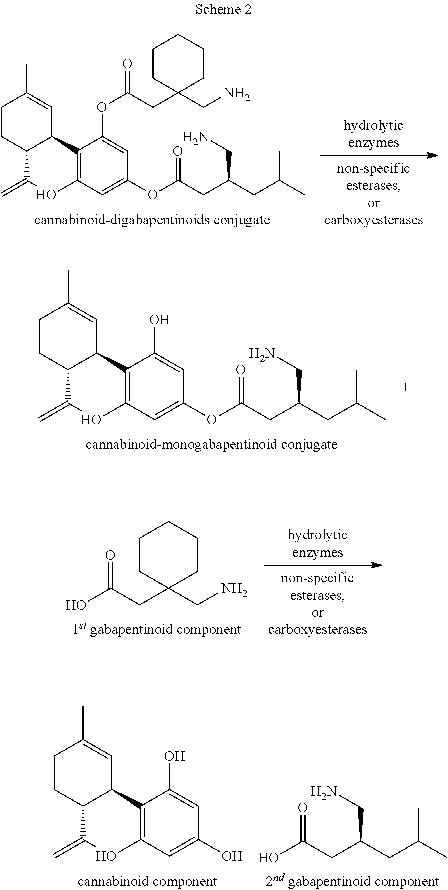 [SMILES: C=C1Oc2cc(OC(=O)C[C@@H](CN)CC(C)C)cc(OC(=O)CC3(CN)CCCCC3)c2[C@@H]2C=C(C)CC[C@@H]12 ; C=C1Oc2cc(OC(=O)C[C@@H](CN)CC(C)C)cc(O)c2[C@@H]2C=C(C)CC[C@@H]12 ; NCC1(CC(=O)O)CCCCC1 ; C=C1Oc2cc(O)cc(O)c2[C@@H]2C=C(C)CC[C@@H]12 ; CC(C)C[C@H](CN)CC(=O)O]

The normal recommended dose for THC is 20 mg/day, while it is 75 mg/day for pregabalin. Therefore, having conjugates with a cannabinoid:gabapentinoid ratio of 1:3 represents an additional advantage of providing the pharmacologically recommended plasma level of both components, after hydrolysis of the conjugate compound.

In certain specific examples, the present invention pertains to conjugates of formulas 12-21 with a cannabinoid:gabapentinoid ratio of 1:3:

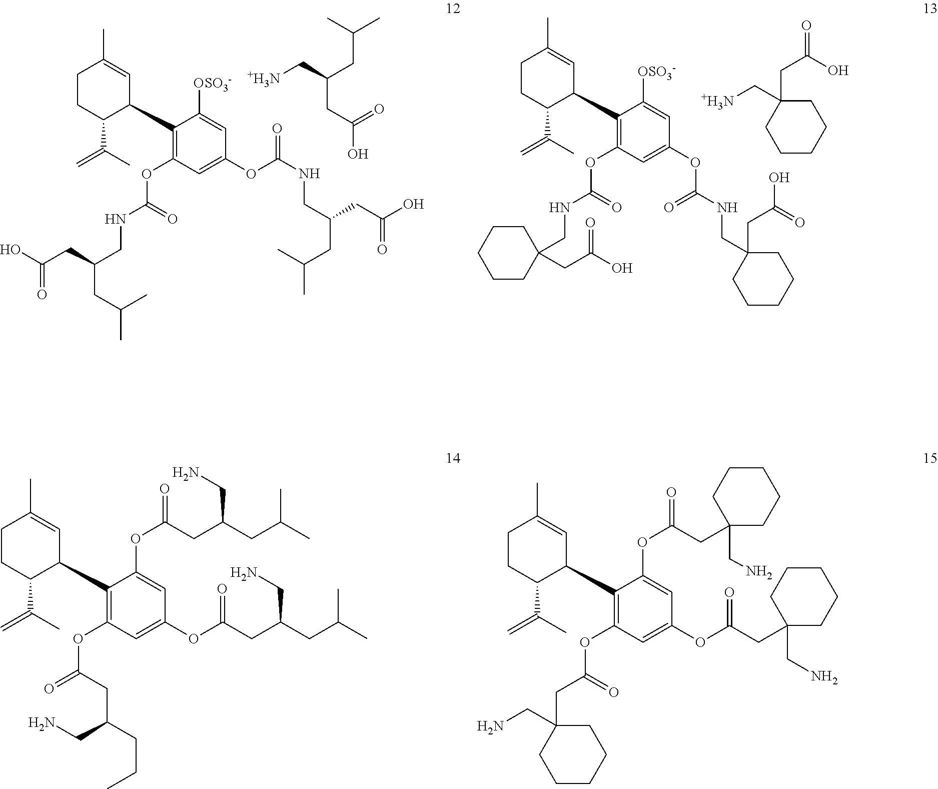

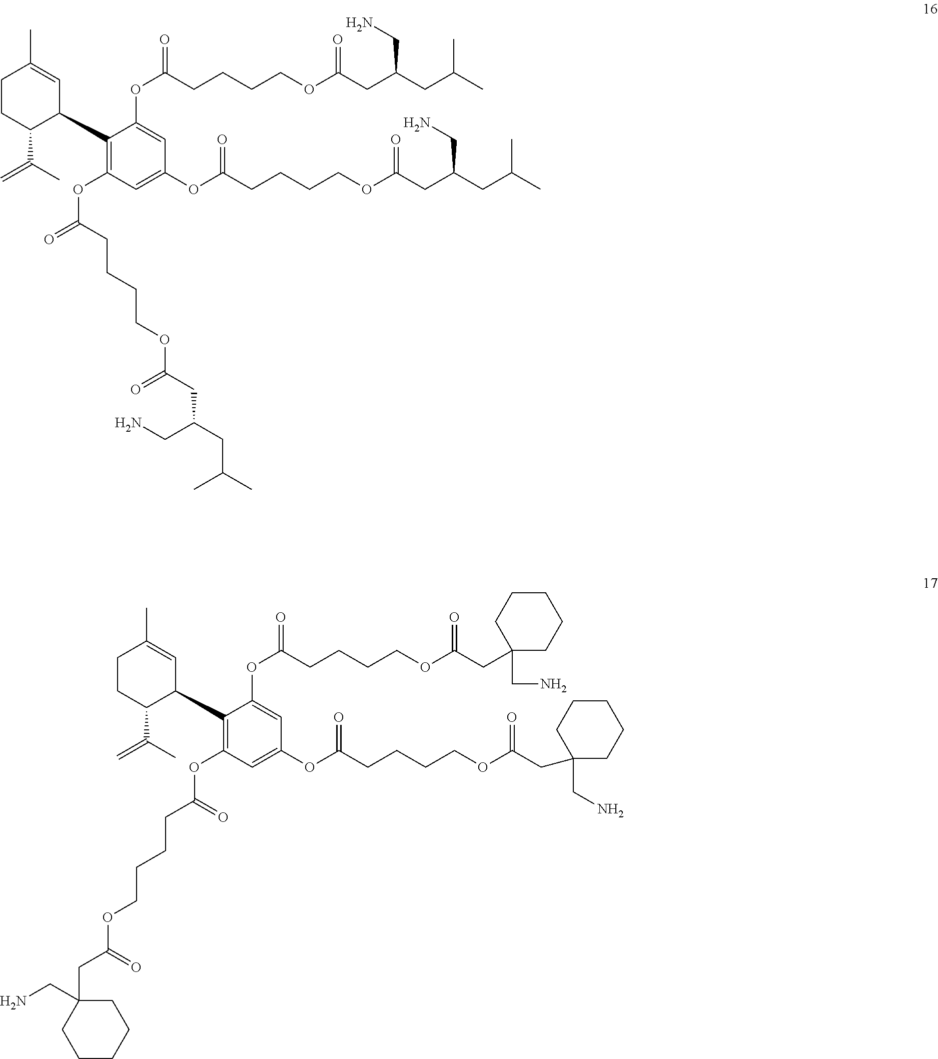 [SMILES: C=C(C)[C@@H]1CCC(C)=C[C@H]1c1c(OC(=O)CCCCOC(=O)C[C@@H](CN)CC(C)C)cc(OC(=O)CCCCOC(=O)C[C@@H](CN)CC(C)C)cc1OC(=O)CCCCOC(=O)C[C@@H](CN)CC(C)C ; C=C(C)[C@@H]1CCC(C)=C[C@H]1c1c(OC(=O)CCCCOC(=O)CC2(CN)CCCCC2)cc(OC(=O)CCCCOC(=O)CC2(CN)CCCCC2)cc1OC(=O)CCCCOC(=O)CC1(CN)CCCCC1]

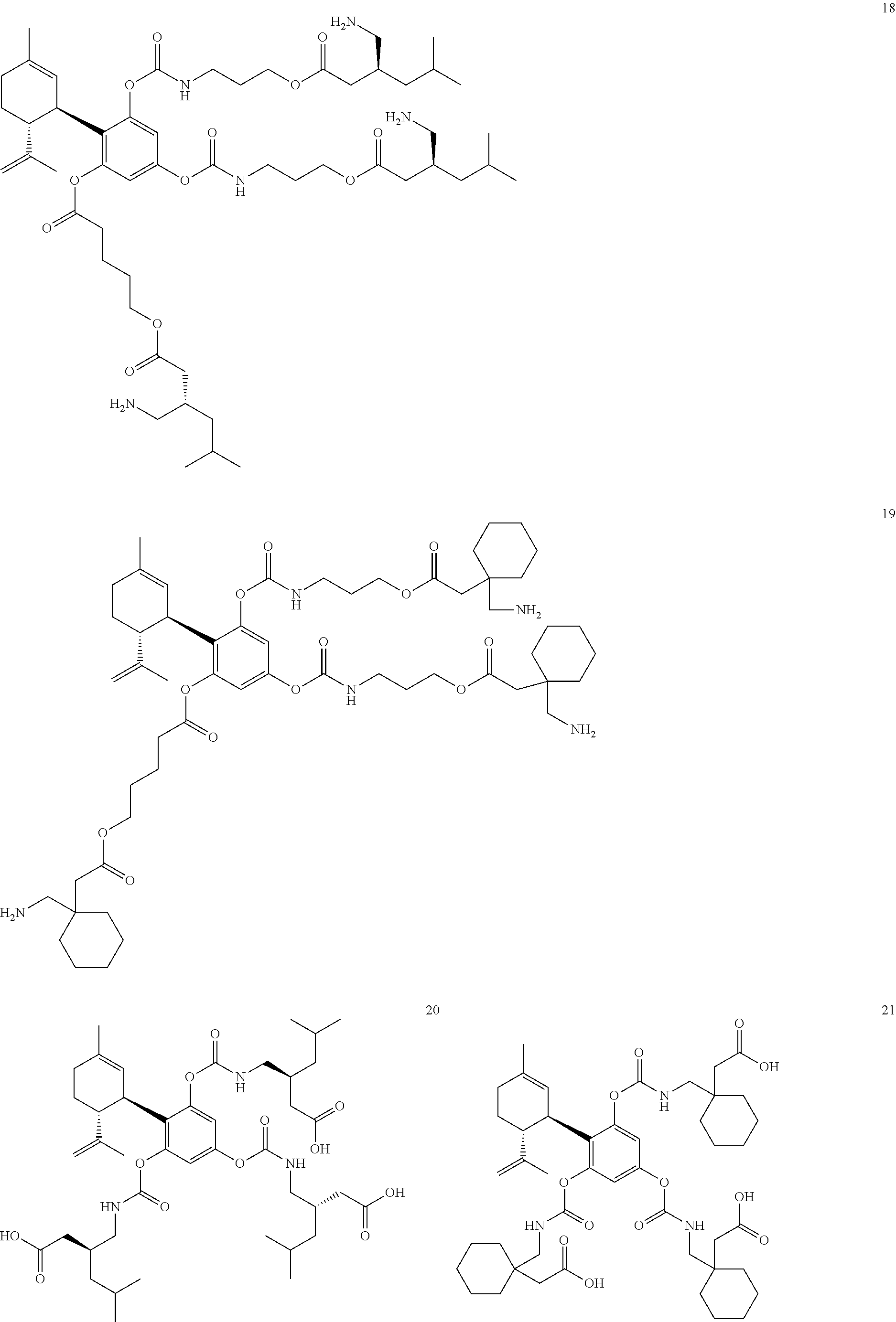

In certain specific examples. this invention pertains to compounds with formulas 22-27, in which the gabapentinoid components are non-identical and the cannabinoid:gabapentinoid ratio is 1:3, or pharmaceutically acceptable salts, hydrates, or solvates thereof:

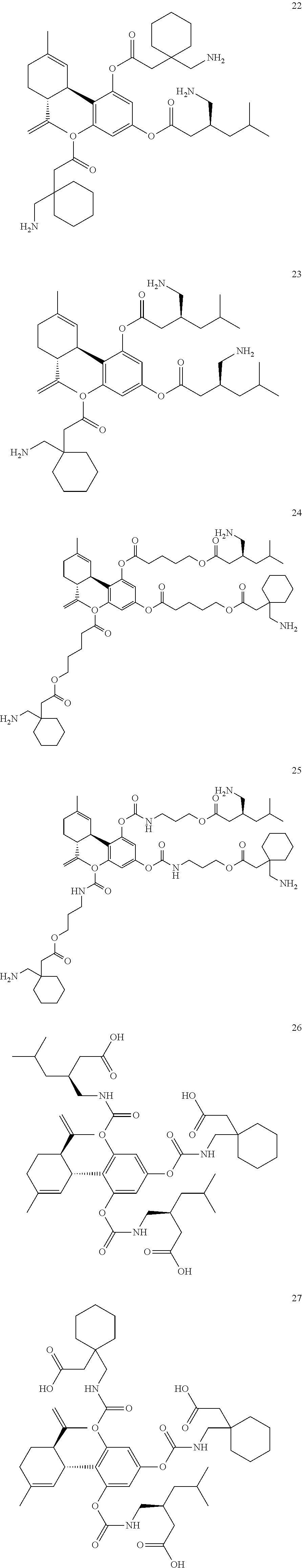

In certain illustrative embodiments, the steric properties of the linker may be modified to have more control on the hydrolysis process or release of active drug moieties. Controlling the steric effect may be achieved by adding one or more substituted or unsubstituted hydrocarbon moieties close to the hydrolysable bond.

In certain specific examples, this invention pertains to compounds with formulas 28-31, in which the linker is sterically modified, or pharmaceutically acceptable salts, hydrates, or solvates thereof:

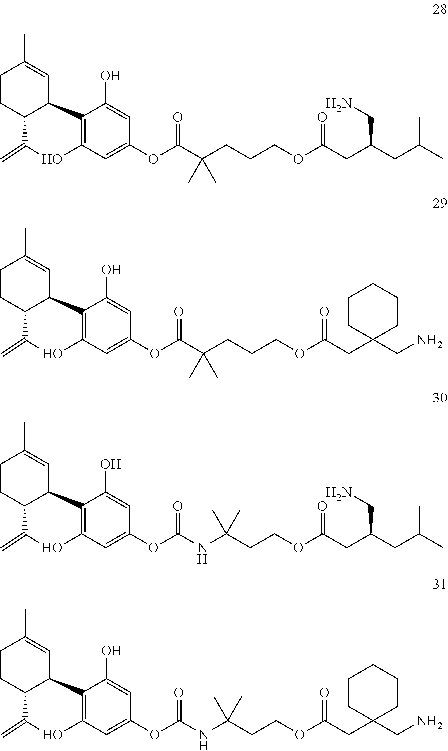 [SMILES: C=C1Oc2cc(OC(=O)C(C)(C)CCCOC(=O)C[C@@H](CN)CC(C)C)cc(O)c2[C@@H]2C=C(C)CC[C@@H]12 ; C=C1Oc2cc(OC(=O)C(C)(C)CCCOC(=O)CC3(CN)CCCCC3)cc(O)c2[C@@H]2C=C(C)CC[C@@H]12 ; C=C1Oc2cc(OC(=O)NC(C)(C)CCOC(=O)C[C@@H](CN)CC(C)C)cc(O)c2[C@@H]2C=C(C)CC[C@@H]12 ; C=C1Oc2cc(OC(=O)NC(C)(C)CCOC(=O)CC3(CN)CCCCC3)cc(O)c2[C@@H]2C=C(C)CC[C@@H]12]

In certain illustrative embodiments, the electronic properties of the linker may be modified to have more control on the hydrolysis process or release of the active drug moieties (i.e. the cannabinoid and gabapentinoid components of the conjugates). Controlling the electronic effect may be achieved by adding electron-withdrawing groups or halides close the hydrolysable bond.

In certain specific examples, this invention pertains to compounds with formulas 32-39, in which the linker is electronically modified, or pharmaceutically acceptable salts, hydrates, or solvates thereof:

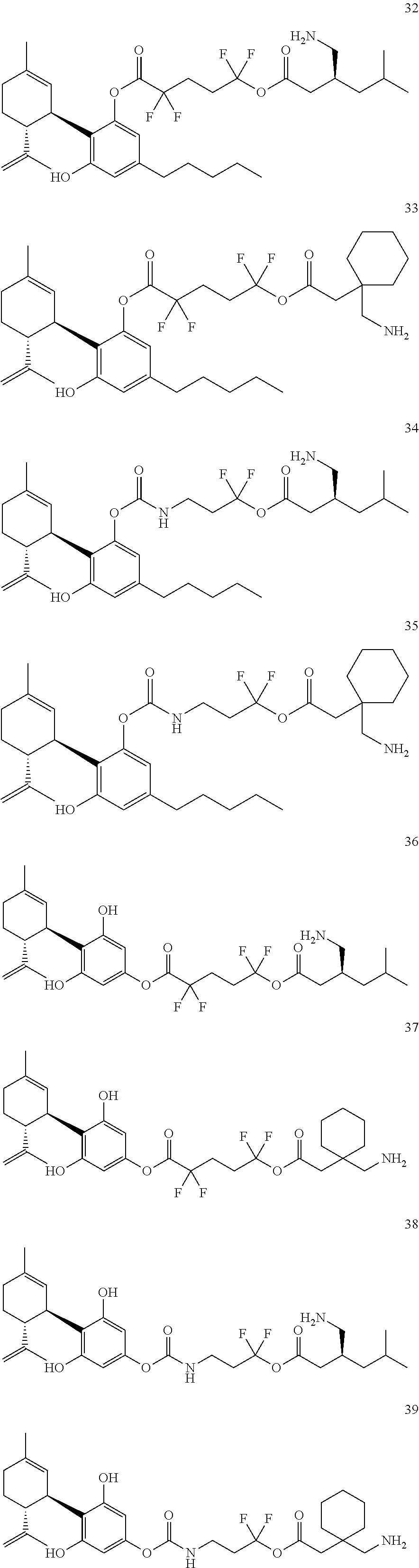

The cannabinoid is a compound that modulates cannabinoid receptor(s), either as agonist, biased agonist, antagonist or with mixed action(s), and may be one or more cannabinoids selected from the group consisting of: delta-9-tetrahydrocannabinol (THC), delta-8-tetrahydrocannabinol (THC), cannabidiol (CBD), cannabinol (CBN), cannabinolic acid (CBNA), cannabigerol (CBG), cannabigerol (CBG), cannabigerovarin (CBGV), cannabichromene (CBC), cannabicyclol (CBL), canabivarol (CBV), tetrahydrocannabivarin (THCV), cannabidivarin (CBDV), cannabichromevarin (CBCV), cannabigerol monoethyl ether (CBGM), cannabigerolic acid monoethyl ether (CBGAM), cannabidiolic acid (CBDA), cannabigerovarinic (CBGVA), cannabichromenic acid (CBCA), cannabichromenic acid (CBCA), cannabidiol monomethylether (CBDM), cannabidiol-C4 (CBD-C4), cannabidivarinic (CBDVA), cannabidiorcol (CBD-C1), delta-9-tetrahydrocannabinolic acid A (THCA-A), delta-9-tetrahydrocannabinolic acid B (THCA-B), delta-9-tetrahydrocannabinolic acid-C4 (THCA-C4), delta-8-tetrahydrocannabinolic acid (delta-8-THCA), delta-8-tetrahydrocannabinol (delta-8-THC), delta-9-tetrahydrocannabinol-C4 (THC-C4), delta-9-tetrahydrocannabiorcolic acid (THCA-C1), delta-9-tetrahydrocannabiorcol-C1 (THC-C1), tetrahydrocannabivarinic acid (THCVA), cannabicycolic acid (CBLA), cannbicyclol (CBL), cannabicyclovarin (CBLV), cannabielsoic acid A (CBEA-A), cannabielsoic acid B (CBEA-B), cannabielsoin (CBE), cannabivarin, cannabinol-C4 (CBN-C4), cannabinol methylether (CBNM), cannabiorcol (CBN-C1), cannabinol-C2 (CBN-C2), cannabinodiol (CBND), cannabinodivarin (CBVD), cannabitriol (CBT), cannabitriolvarin (CBTV), dehydrocannabifuran (DCBF), cannabifuran, cannabicitran (CBT), cannabiripsol (CBR), ‘11-hydroxytetrahydrocannabinol’ (11-OH-THC), ‘11-nor-9-carboxy-tetrahydrocannabinol’ (THC-COOH), and their derivatives, synthetic analogues, related chemical structures and salts, and mixtures and combinations thereof. Preferably, the cannabinoid is CBD, THC, CBDA, THCA or THCV.

In another embodiment, the cannabinoid may be a metabolite of any of the cannabinoids listed above. Preferred examples of natural and synthetic cannabinoids are:

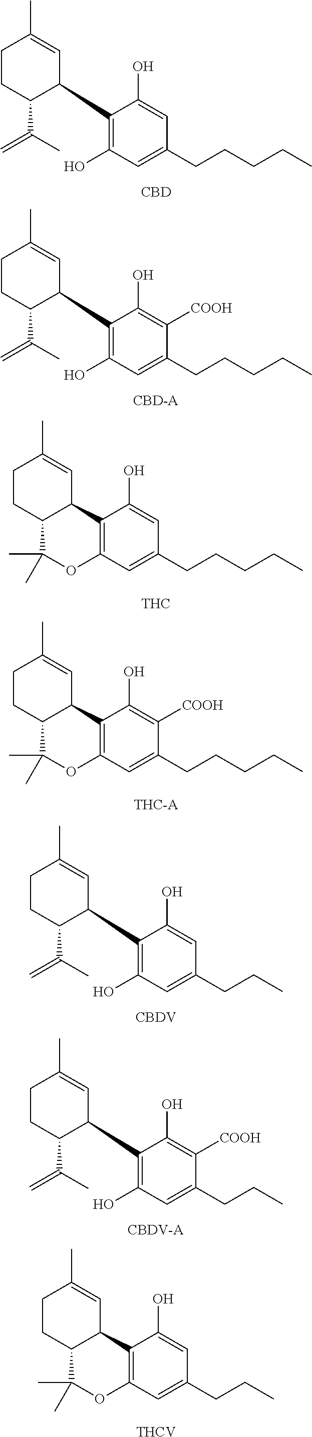

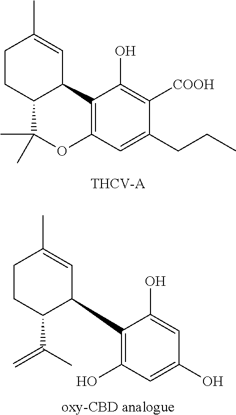

In another embodiment, the gabapentinoid is one or more gabapentinoids selected from the group consisting of: GABA, gabapentin, pregabalin, Phenibut, tolgabide, progabide, picamilon, γ-amino-β-hydroxybuteric acid, cis-2-Aminomethylcyclopropane carboxylic acid, (Z)-4-Amino-2-butenoic acid, Lesogaberan, γ-valerolactone, γ-hydroxyvaleric acid, γ-hydroxybutyric acid, γ-butyrolactone, baclofen, and gabamide.

In another embodiment, the gabapentinoid may be a metabolite of any of the gabapentinoids listed above. Preferred examples of gabapentinoids are:

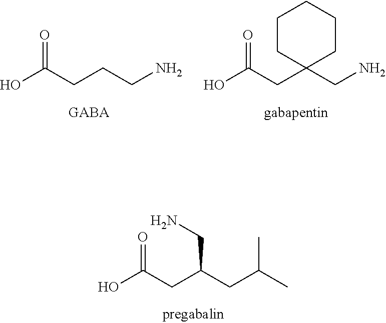

In another embodiment, the linker may be covalent or ionic in nature. Covalent linkers may be linear, cyclic or branched alkyl carbon chain, functionalized or not, with different functionalities such as ester, amide, acetal, ketal, amino acid, short peptide, phosphate, phosphonate, each of which is optionally substituted. Ionic linkers include salts of carboxylates, phosphates, phosphonates, sulfates, sulfonates, sulfamates and related structures.

In another embodiment, the linker releases the cannabinoid and gabepentinoid components of the conjugate in the body of the subject in need when subjected to metabolic enzymes, or chemical hydrolysis.

In any of the embodiments described in formula I, in which two or more gabapentinoid components may be attached, each gabapentinoid component may be the same or different, and, when linkers are used, each linker may be the same or different.

The compounds described herein may be used alone or in combination with other compounds that may be therapeutically effective by the same or different modes of action. In addition, the compounds described herein may be used in combination with other compounds that are administered to treat other symptoms of psychiatric disorders, such as compounds administered to relieve pain, nausea, vomiting, and the like.

In some other embodiments, this invention pertains to a pharmaceutical composition comprising a compound disclosed herein, or a pharmaceutically acceptable salt thereof, together with one or more pharmaceutically acceptable diluents or excipients.

In some embodiments, this invention pertains to a pharmaceutical composition comprising a compound disclosed herein, in combination with one or more other therapeutically active compounds by the same or different mode of action, and one or more pharmaceutically acceptable diluents or excipients.

In some other embodiments, cannabinoid moiety is a modulator of the endocannabinoid system, for example, of cannabinoid receptor CB1, CB2 and other related molecular targets, while the gabapentinoid component exerts its pharmacological effect via a different and complex mechanism.

In some embodiments, modulators pertain to allosteric modulators, agonist, biased agonist, antagonist, biased antagonist or partial agonist of cannabinoid receptor(s), blocking the reuptake of serotonin, modulating the level of neurotransmitters in CNS or peripheral tissues, modulating the level of cellular secondary messengers or modulating the phosphorylated level of cellular enzymes or proteins.

In some embodiments, this invention pertains to a method for treating a patient of epilepsy, neuropathic pain, multiple sclerosis, seizures and postherpetic neuralgia, restless leg syndrome, trigeminal neuralgia, fibromyalgia, diabetic neuropathy, anxiety and bipolar disorders, schizophrenia, sleep disorders, post-traumatic stress disorder, anorexia (which may be related to cancer or HIV infection), movement disorders, Tourette syndrome, glaucoma, traumatic brain injury and Crohn disease, chronic pain and spasticity, nausea and vomiting (due to chemotherapy), weight gain (in HIV infection), postherpetic neuralgia, migraine, social phobia, panic disorder, mania, alcohol withdrawal, and other related psychiatric disorders and pathological conditions. The method comprising the step of administering a therapeutically effective amount of a compound disclosed herein, together with one or more pharmaceutically acceptable diluents, and excipients, to the patient in need of relief from said psychological disorder(s).

In some embodiments, this invention pertains to a pharmaceutical composition comprising a compound disclosed herein, in combination with one or more other therapeutically active compounds by the same or different mode of action, and one or more pharmaceutically acceptable diluents, and excipients.

### Examples

### Example 1

In one exemplary embodiment, a cannabinoid-gabapentinoid conjugate may be prepared according to the following method. Boc-protected pregabalin (1 equiv.) is activated using 1,1′-carbonyldiimidazole (CDI) (1 equiv.), and then the CBD is added. The reaction mixture is stirred at 80° C. for 12 hours. Progress of the reaction may be monitored by TLC. After complete conversion, the reaction is quenched with distilled water (50 mL) and organic material is extracted with ethyl acetate (50 mL×3), collected, dried over anhydrous MgSO₄, and concentrated under reduced pressure. The crude intermediate is dissolved in absolute DMF (20 mL), charged with TFA (2 mL), and heated at 80° C. for 2 hours. After the reaction is completion, it is quenched with distilled water (50 mL) and organic material is again extracted with ethyl acetate (50 mL×3), collected, dried over anhydrous MgSO₄, and concentrated under reduced pressure. The cannabinoid-gabapentinoid conjugate is then purified by normal phase column chromatography using hexane:ethyl acetate (4:1) (Scheme 4).

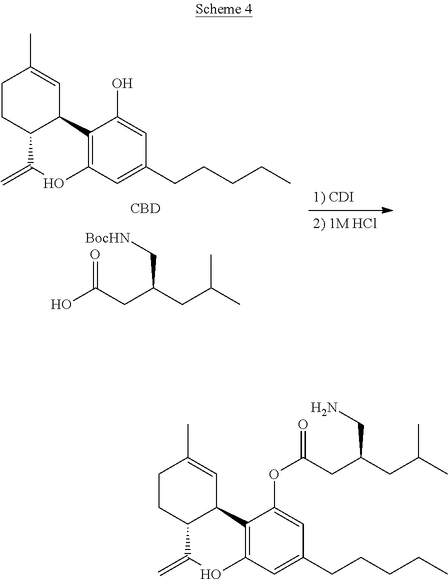

### Example 2

In another exemplary embodiment, a cannabinoid-gabapentinoid conjugate may be prepared according to the following method. THC-V (1 equiv.) and fluorinated lactone (1 equiv.) are dissolved in dry acetone (about 25 mL). Potassium carbonate (3 equiv.) is added to the reaction mixture and the reaction is heated to 50° C. After the reaction is completion, it is quenched with distilled water (about 50 mL) and organic material is extracted with ethyl acetate (about 50 mL×3), collected, dried over anhydrous MgSO₄, and concentrated under reduced pressure. The crude product is dissolved in dry DMF, CDI-activated Boc-protected pregabalin (1 equiv.) is added, and the reaction mixture is stirred at 80° C. for 12 hours. Progress of the reaction may be monitored by TLC. After the reaction is complete, it is quenched with distilled water (about 50 mL) and organic material is again extracted with ethyl acetate (about 50 mL×3), collected, dried over anhydrous MgSO₄, and concentrated under reduced pressure. The cannabinoid-gabapentinoid conjugate is then purified by normal phase column chromatography using hexane:ethyl acetate (4:1) (Scheme 5).

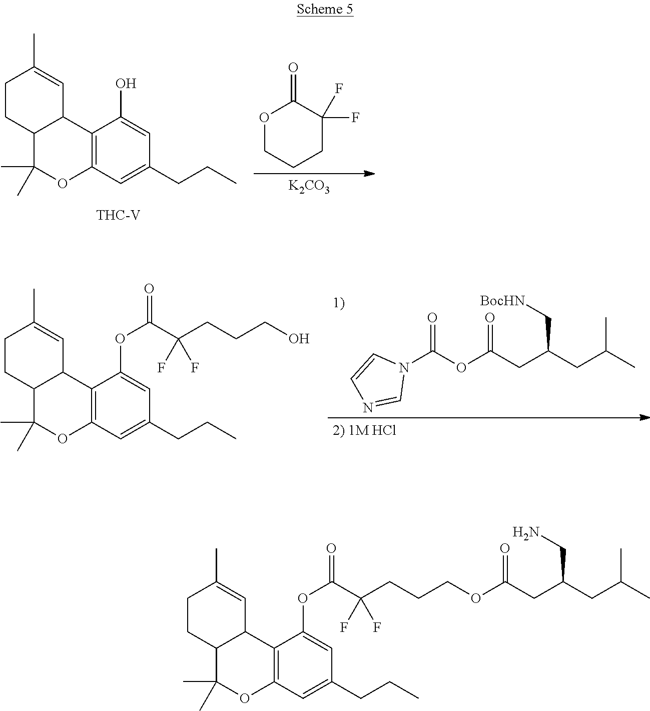

In other embodiments, the 1,1′-carbonyldiimidazole (CDI) may be replaced by other coupling reagents including: phosgene, trichloroacetyl chloride, 1,1′-carbonylbis (2-methylimidazole), N,N′-disuccinimidyl carbonate, 4-nitrophenylchloroformate, and bis(4-nitrophenyl)carbonate, bis(pentafluorophenyl)carbonate.

### Molecular Docking

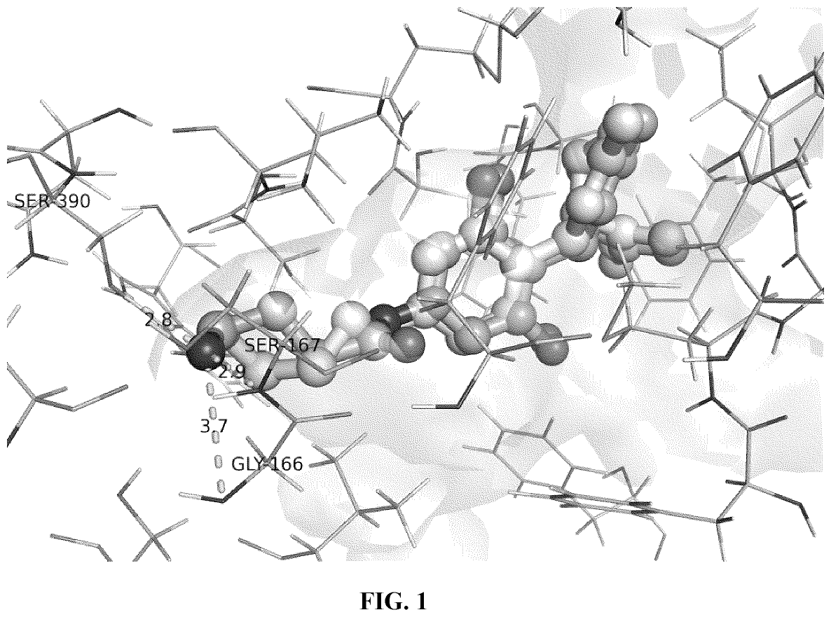

Compound 1a, described above, was taken as an exemplary representative compound. The calculated binding pose of compound 1a was found to be perfectly overlapped with CBD, as shown in the below figure. Furthermore, the terminal amino group was calculated to be docked within polar residues, in which it was calculated to interact strongly with the hydroxyl group of Ser390, and the amino group of Ser167, through two strong H-bonds as shown in FIG. 1.

FIG. 1 illustrates the calculated binding poses of CBD (C-backbone colored gray), and the CBD-pregabalin conjugate of example compound 1a (C-backbone colored light-red) within the pocket of CB1 receptor (PDB ID: 5U09). Only residues within 4A were shown for the sake of clarity. Nitrogen atoms are colored blue, and oxygen atoms are colored red. All hydrogen atoms from both compounds are removed for the sake of clarity. Distances are calculated based on heavy atoms.

As used herein, the following terms and phrases shall have the meanings set forth below. Unless defined otherwise, all technical and scientific terms used herein have the same meaning as commonly understood to one of ordinary skill in the art.

As used herein, the term “about” can allow for a degree of variability in a value or range, for example, within 1%, within 5%, or within 10% of a stated value or of a stated limit of a range. In the present disclosure the term “substantially” can allow for a degree of variability in a value or range, for example, within 90%, within 95%, 99%, 99.5%, 99.9%, 99.99%, or at least about 99.999% or more of a stated value or of a stated limit of a range.

As used herein, the term “alkyl” refers to a saturated monovalent chain of carbon atoms, which may be optionally branched. It is understood that in embodiments that include alkyl, illustrative variations of those embodiments include lower alkyl, such as C₁ to C₉ alkyl, methyl, ethyl, propyl, 3-methylbutyl, and the like. It is understood that each of alkyl moieties may be optionally substituted with independently selected groups such as halide, alkyl, alkoxy, hydroxy, hydroxyalkyl, carboxylic acid and derivatives thereof, including esters, nitrile, amides, and nitrites, acyloxy, aminoalkyl and dialkylamino, acylamino, thio, and the like, and combinations thereof.

The term “optionally substituted,” or “optional substituents,” as used herein, means that the groups in question are either unsubstituted or substituted with one or more of the substituents specified. When the groups in question are substituted with more than one substituent, the substituents may be the same or different. Moreover, when using the term “independently”, this means that the groups in question may be the same or different. Certain of the herein defined terms may occur more than once in the structure, and upon such occurrence each term shall be defined independently of the other, unless defined otherwise.

The term “subject” or “patient” includes human and non-human animals such as companion animals such as dogs and cats and the like, and livestock animals. Livestock animals are animals raised for food production. The subject/patient to be treated is preferably a mammal, in particular, a human being.

The term “pharmaceutically acceptable diluent” or “pharmaceutically acceptable excipient” is art-recognized and refers to a pharmaceutically acceptable material, composition or vehicle, such as a liquid or solid filler, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose and maltose; (2) starches, such as corn starch and gelatinized starch; (3) cellulose, and its derivatives, such as carboxymethyl cellulose salt, and hydroxypropylmethyl cellulose; (4) thickening agents such as gelatin and tragacanth; (5) disintegrants such as copovidone; (6) other excipients, such as cocoa butter and suppository waxes and pyrogen-free water for sterile products; and (7) other non-toxic compatible substances employed in pharmaceutical formulations.

As used herein, the term “administering” includes all means of introducing the compounds and compositions described herein to the patient, including, but are not limited to, topical, oral, intravenous, intramuscular, transdermal, inhalation, buccal, ocular, vaginal, rectal, and the like. The compounds and compositions described herein may be administered in unit dosage forms or formulations containing conventional nontoxic pharmaceutically acceptable carriers, adjuvants, and vehicles.

The present invention has been described and illustrated with reference to an exemplary embodiment; however, it will be understood by those skilled in the art that various changes may be made, and equivalents may be substituted for elements thereof without departing from the scope of the invention as set out in the following claims. Therefore, it is intended that the invention is not limited to the embodiments disclosed herein.

What is claimed is:

1. A compound of formula I, or a pharmaceutically acceptable salt, hydrate, or solvate thereof: 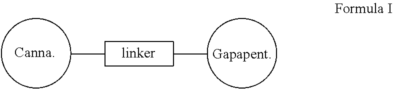
wherein:
Canna is a cannabinoid that modulates one or more cannabinoid receptors, either as an agonist, biased agonist, antagonist, or with mixed actions,
Gabapent is a gabapentinoid with a carboxylic acid functionality or its isostere or analogues connected through a three-carbon chain to an amino group or its isostere or analogues,
linker is selected from the group consisting of: a hydrolysable covalent or ionic bond, or a linear, cyclic, or branched alkyl carbon chain, which is optionally functionalized or substituted or both, and is attached independently to the cannabinoid and gabapentinoid by a hydrolysable covalent or ionic bond; and
wherein the cannabinoid may have one or more gabapentinoids each attached by a linker.

2. The compound of claim 1, wherein each of the one or more gabapentinoids is independently a compound of formula II: 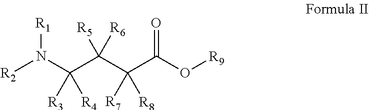
wherein:
R₁ and R₂ are independently selected from the group consisting of: H, and alkyl group, a substituted alkyl group, an aceyl group, or a substituted aceyl group,
R₃ to R₈ are independently selected from the group consisting of: H, a halogen, a nitrile group, hydroxide, an alkyl group, a substituted alkyl group, a cyclic alkyl group, a substituted cyclic alkyl group, an alkoxy group, and alkenyl group, a substituted alkenyl group, a cyclic alkenyl group, or a substituted cyclic alkenyl group,
R₉ is H or an alkyl group, and
wherein each of R₁ to R₉ may be part of a cyclic structure with another of R₁ to R₉.

3. The compound of claim 2, wherein each of the one or more gabapentinoids is independently selected from the group consisting of: GABA, gabapentin, pregabalin, Phenibut, tolgabide, progabide, picamilon, γ-amino-b-hydroxy buteric acid, cis-2-Aminomethylcyclopropane carboxylic acid, (Z)-4-Amino-2-butenoic acid, Lesogaberan, γ-valerolactone, γ-hydroxyvaleric acid, γ-hydroxybutyric acid, γ-butyrolactone, baclofen, and gabamide.

4. The compound of claim 3, wherein each of the one or more gabapentinoids is independently selected from the group consisting of: GABA, pregabalin, and gabapentin.

5. The compound of claim 4, wherein the cannabinoid is selected from the group consisting of: delta-9-tetrahydrocannabinol (THC), delta-8-tetrahydrocannabinol (THC), cannabidiol (CBD), cannabinol (CBN), cannabinolic acid (CBNA), cannabigerol (CBG), cannabigerol (CBG), cannabigerovarin (CBGV), cannabichromene (CBC), cannabicyclol (CBL), canabivarol (CBV), tetrahydrocannabivarin (THCV), cannabidivarin (CBDV), cannabichromevarin (CBCV), cannabigerol monoethyl ether (CBGM), cannabigerolic acid monoethyl ether (CBGAM), cannabidiolic acid (CBDA), cannabigerovarinic (CBGVA), cannabichromenic acid (CBCA), cannabichromenic acid (CBCA), cannabidiol monomethylether (CBDM), cannabidiol-C4 (CBD-C4), cannabidivarinic (CBDVA), cannabidiorcol (CBD-C1), delta-9-tetrahydrocannabinolic acid A (THCA-A), delta-9-tetrahydrocannabinolic acid B (THCA-B), delta-9-tetrahydrocannabinolic acid-C4 (THCA-C4), delta-8-tetrahydrocannabinolic acid (delta-8-THCA), delta-8-tetrahydrocannabinol (delta-8-THC), delta-9-tetrahydrocannabinol-C4 (THC-C4), delta-9-tetrahydrocannabiorcolic acid (THCA-C1), delta-9-tetrahydrocannabiorcol-C1 (THC-C1), tetrahydrocannabivarinic acid (THCVA), cannabicycolic acid (CBLA), cannbicyclol (CBL), cannabicyclovarin (CBLV), cannabielsoic acid A (CBEA-A), cannabielsoic acid B (CBEA-B), cannabielsoin (CBE), cannabivarin, cannabinol-C4 (CBN-C4), cannabinol methylether (CBNM), cannabiorcol (CBN-C1), cannabinol-C2 (CBN-C2), cannabinodiol (CBND), cannabinodivarin (CBVD), cannabitriol (CBT), cannabitriolvarin (CBTV), dehydrocannabifuran (DCBF), cannabifuran, cannabicitran (CBT), cannabiripsol (CBR), ‘11-hydroxytetrahydrocannabinol’ (11-OH-THC), ‘11-nor-9-carboxy-tetrahydrocannabinol’ (THC-COOH), and derivatives, synthetic analogues, and salts thereof.

6. The compound of claim 5, wherein the cannabinoid is selected from the group consisting of: CBD, CBDA, THC, THCA, CBDV, CBDVA, THCV, THCVA, and oxy-CBD analogue.

7. The compound of claim 5, wherein the cannabinoid is selected from the group consisting of: CBD, THC, CBDA, THCA, and THCV.

8. The compound of claim 1, wherein the compound is a compound of formula 1 or a pharmaceutically acceptable salt, hydrate, or solvate thereof: 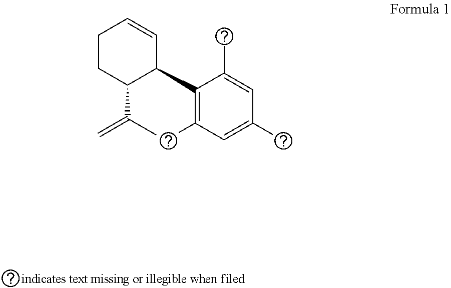
wherein:
R₁ is selected from the group consisting of: 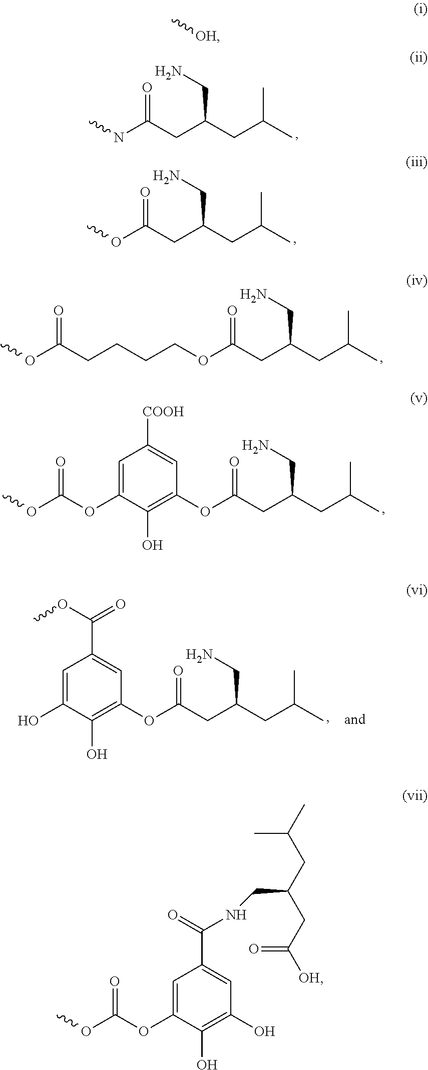
R₂ is selected from the group consisting of: (i) propyl, (ii) pentyl, (iii) heptyl, 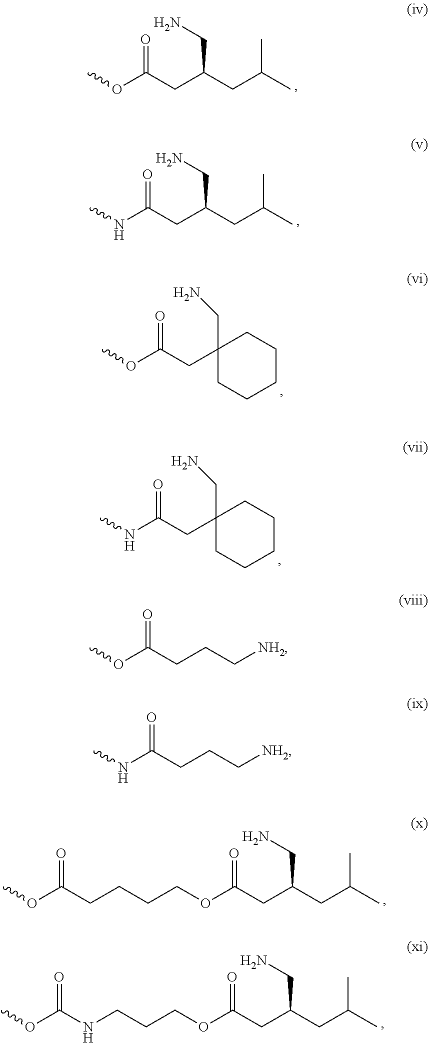 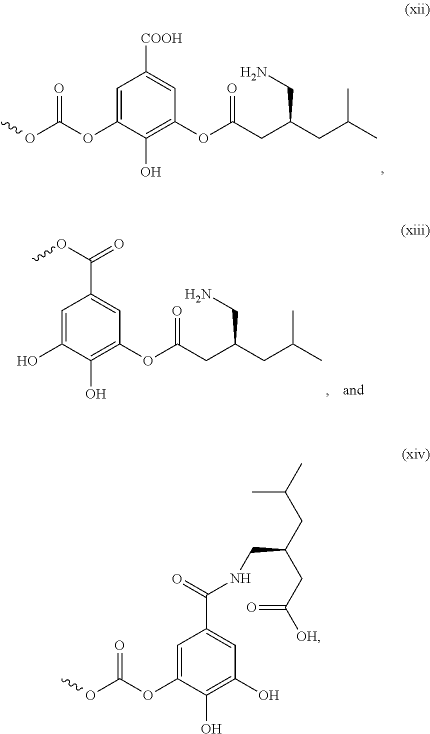
and
R₃ is selected from the group consisting of:

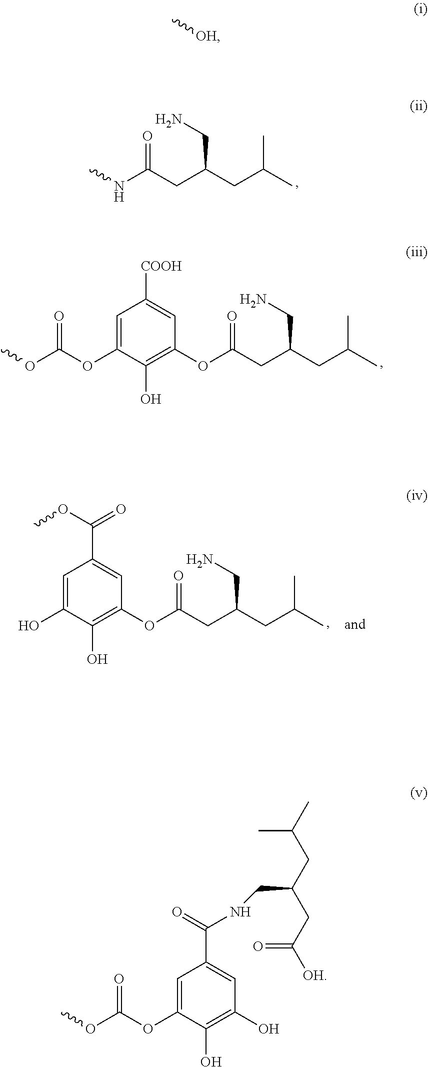

9. The compound of claim 8, wherein R₁ and R₃ are 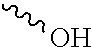
and R₂ is

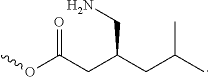

10. The compound of claim 8, wherein R₁ and R₃ are 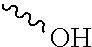
and R₂ is

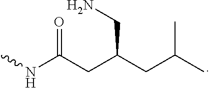

11. The compound of claim 8, wherein R₁ and R₃ are 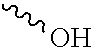
and R₂ is

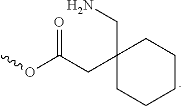

12. The compound of claim 8, wherein R₁ and R₃ are 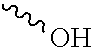
and R₂ is

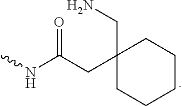

13. The compound of claim 8, wherein R₁ and R₃ are 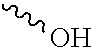
and R₂ is

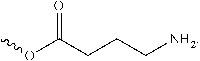

14. The compound of claim 1, wherein the compound is a compound of formula 2 or a pharmaceutically acceptable salt, hydrate, or solvate thereof: 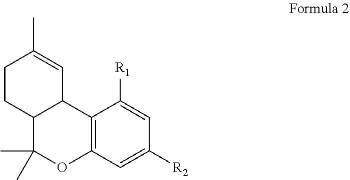
wherein:
R₁ is selected from the group consisting of: 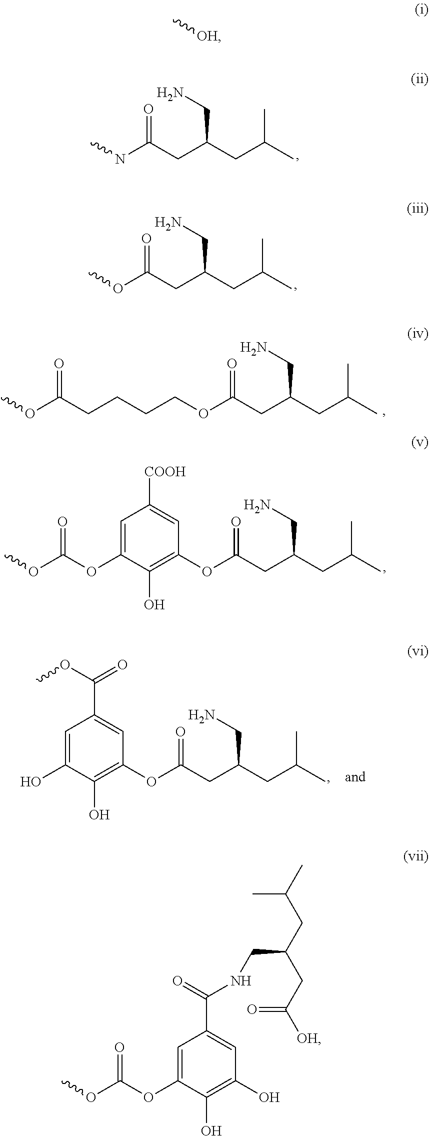
and
R₂ is selected from the group consisting of: (i) propyl, (ii) pentyl, (iii) heptyl,

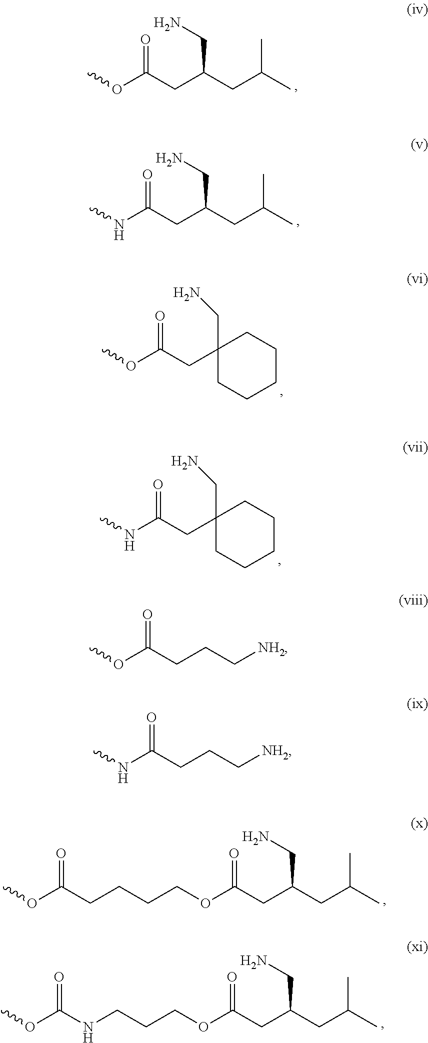

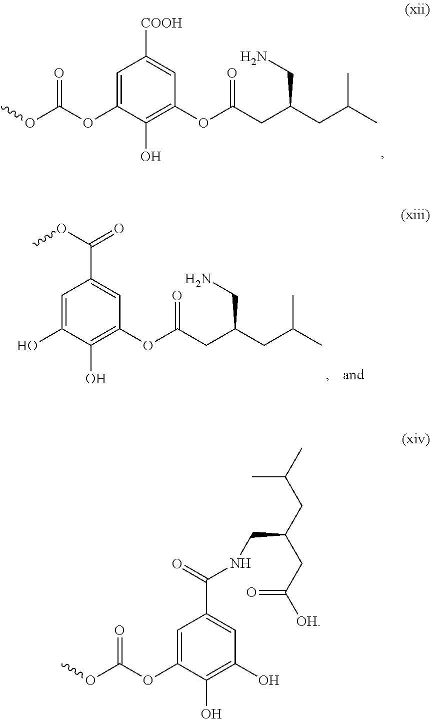

15. The compound of claim 14, wherein R₁ and R₃ are 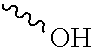
and R₂ is

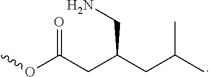

16. The compound of claim 14, wherein R₁ and R₃ are 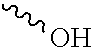
and R₂ is

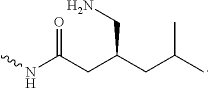

17. The compound of claim 14, wherein R₁ and R₃ are 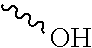
and R₂ is

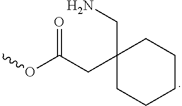

18. The compound of claim 14, wherein R₁ and R₃ are 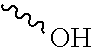
and R₂ is

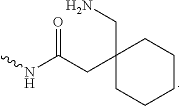

19. The compound of claim 14, wherein R₁ and R₃ are 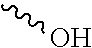
and R₂ is

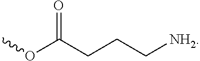

20. The compound of claim 1, wherein the compound is a compound of formula 3 or a pharmaceutically acceptable salt, hydrate, or solvate thereof: 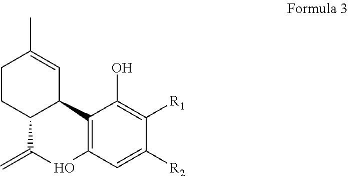
wherein:
R₁ is selected from the group consisting of: (i) COOH, 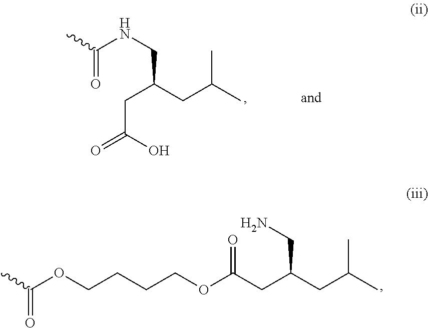
and
R₂ is selected from the group consisting of: (i) propyl, (ii) pentyl,

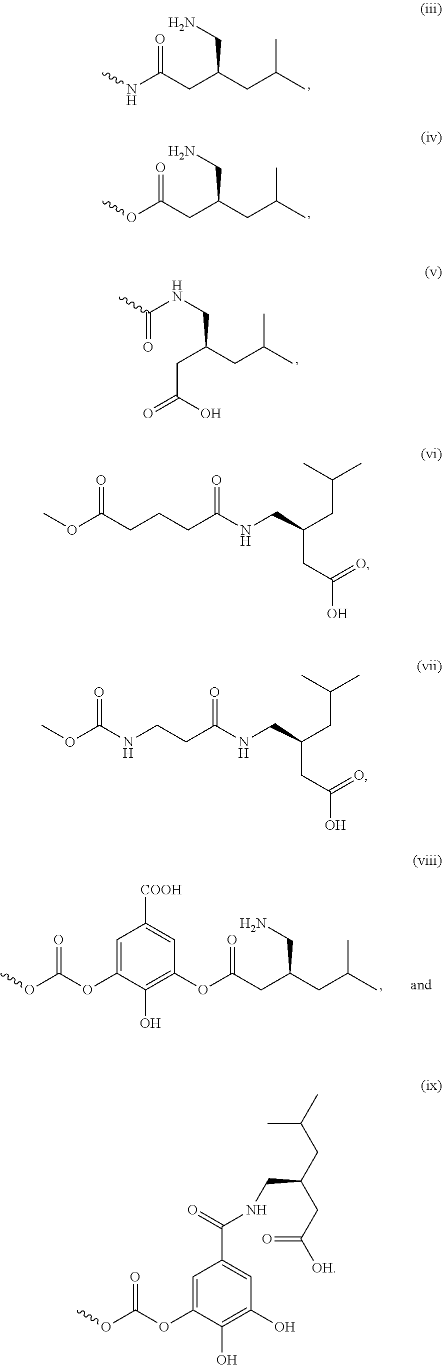

21. The compound of claim 20, wherein R₁ is 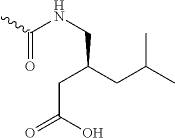
and R₂ is propyl.

22. The compound of claim 20, wherein R₁ is 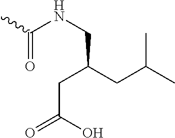
and R₂ is pentyl.

23. The compound of claim 1, wherein the compound is a compound of formula 4 or a pharmaceutically acceptable salt, hydrate, or solvate thereof: 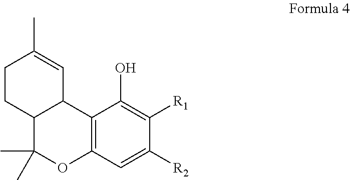
wherein:
R₁ is selected from the group consisting of: (i) COOH, 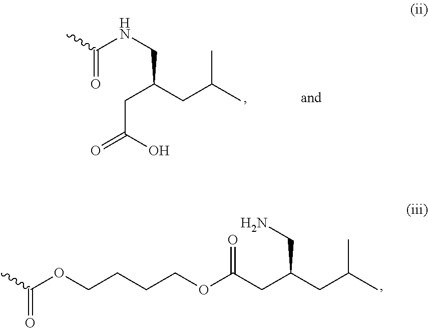
and
R₂ is selected from the group consisting of: (i) propyl, (ii) pentyl,

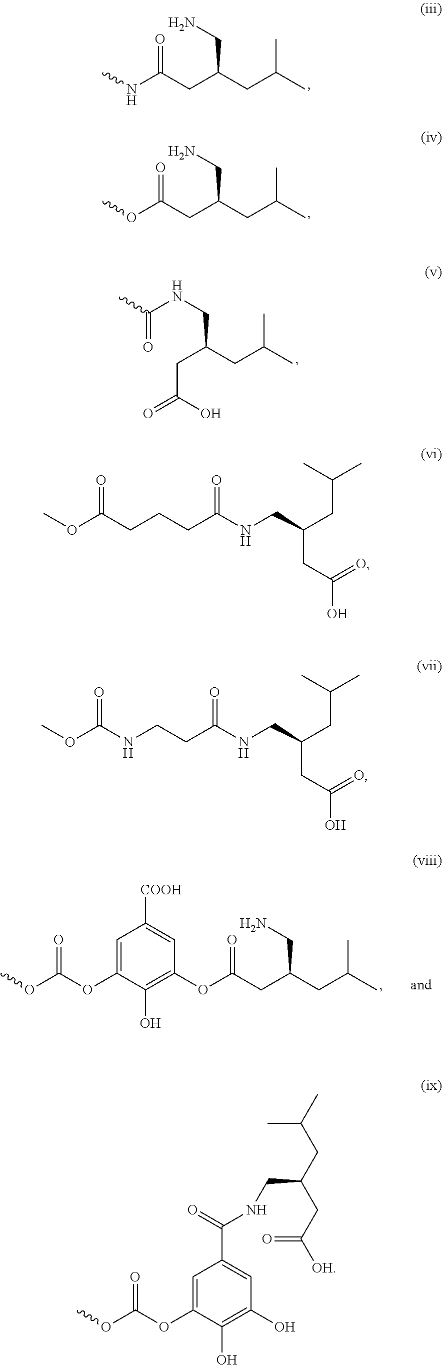

24. The compound of claim 23, wherein R₁ is 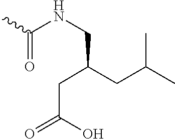
and R₂ is propyl.

25. The compound of claim 23, wherein R₁ is 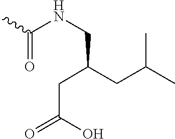
and R₂ is pentyl.

26. The compound of claim 1, wherein the compound is a compound of formula 5 or a pharmaceutically acceptable salt, hydrate, or solvate thereof: 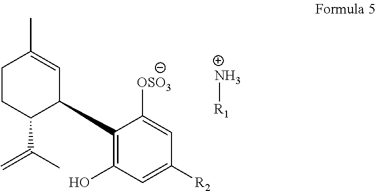
wherein:
R₁ is selected from the group consisting of: 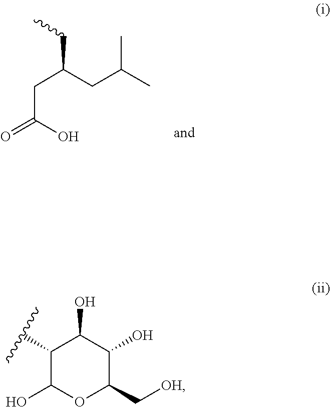
and
R₂ is selected from the group consisting of: (i) propyl, pentyl,

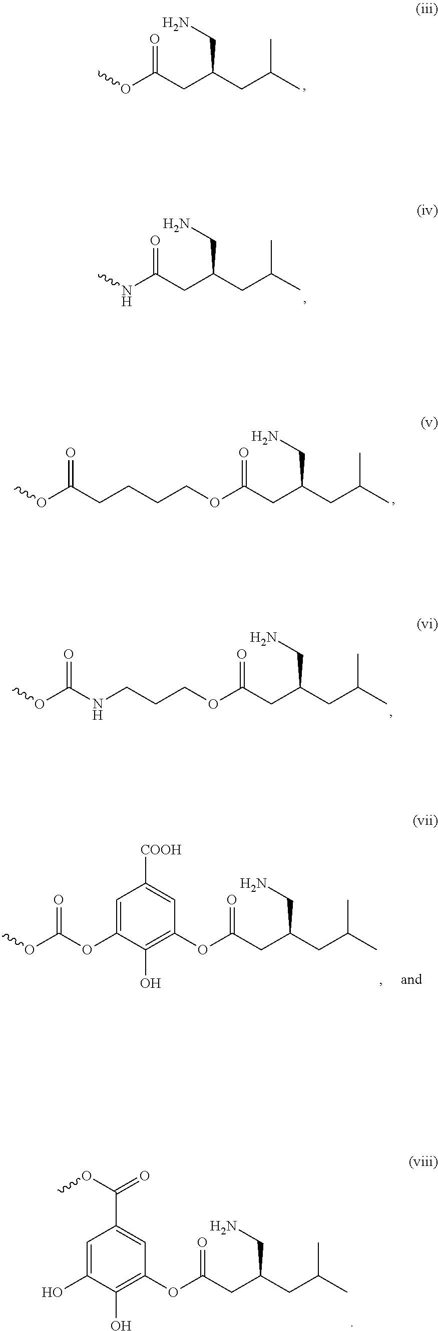

27. The compound of claim 26, wherein R₁ is 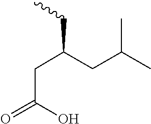
and R₂ is propyl.

28. The compound of claim 26, wherein R₁ is 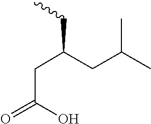
and R₂ is pentyl.

29. The compound of claim 1, wherein the compound is a compound of formula 6 or a pharmaceutically acceptable salt, hydrate, or solvate thereof: 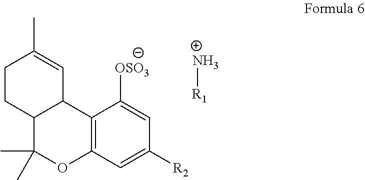
wherein:
R₁ is selected from the group consisting of: 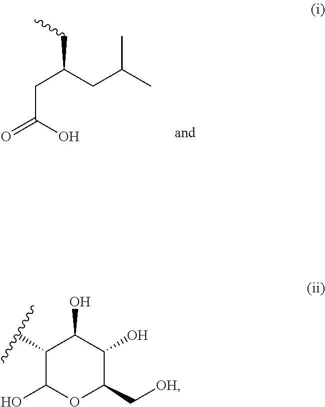
and
R₂ is selected from the group consisting of: (i) propyl, (ii) pentyl, (iii)

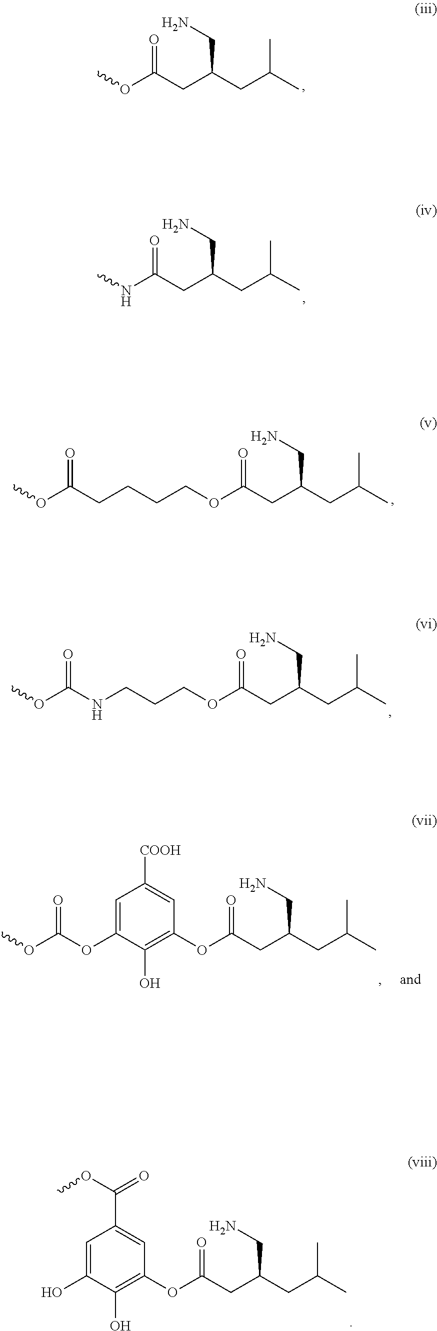

30. The compound of claim 29, wherein R₁ is 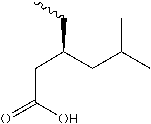
and R₂ is propyl.

31. The compound of claim 29, wherein R₁ is 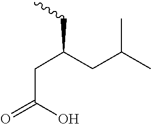
and R₂ is pentyl.

32. The compound of claim 1, wherein the compound is a compound of formula 7 or a pharmaceutically acceptable salt, hydrate, or solvate thereof:

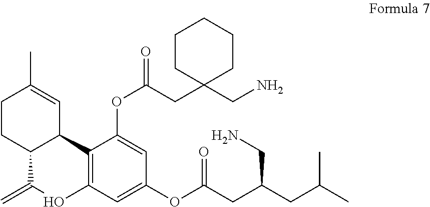 [SMILES: C=C1Oc2cc(OC(=O)C[C@@H](CN)CC(C)C)cc(OC(=O)CC3(CN)CCCCC3)c2[C@@H]2C=C(C)CC[C@@H]12]

33. The compound of claim 1, wherein the compound is a compound of formula 8 or a pharmaceutically acceptable salt, hydrate, or solvate thereof:

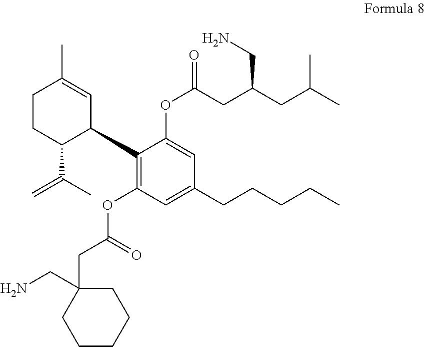 [SMILES: C=C(C)[C@@H]1CCC(C)=C[C@H]1c1c(OC(=O)C[C@@H](CN)CC(C)C)cc(CCCCC)cc1OC(=O)CC1(CN)CCCCC1]

34. The compound of claim 1, wherein the compound is a compound of formula 9 or a pharmaceutically acceptable salt, hydrate, or solvate thereof:

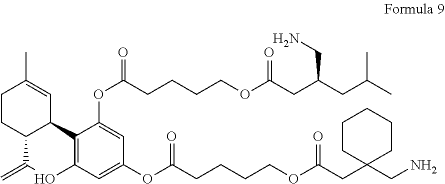 [SMILES: C=C1Oc2cc(OC(=O)CCCCOC(=O)CC3(CN)CCCCC3)cc(OC(=O)CCCCOC(=O)C[C@@H](CN)CC(C)C)c2[C@@H]2C=C(C)CC[C@@H]12]

35. The compound of claim 1, wherein the compound is a compound of formula 10 or a pharmaceutically acceptable salt, hydrate, or solvate thereof:

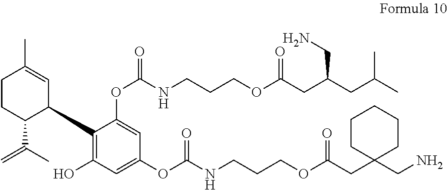 [SMILES: C=C(C)[C@@H]1CCC(C)=C[C@H]1c1c(O)cc(OC(=O)NCCCOC(=O)CC2(CN)CCCCC2)cc1OC(=O)NCCCOC(=O)C[C@@H](CN)CC(C)C]

36. The compound of claim 1, wherein the compound is a compound of formula 11 or a pharmaceutically acceptable salt, hydrate, or solvate thereof:

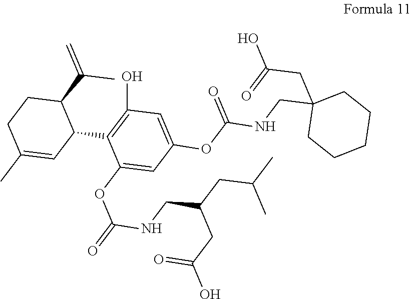

37. The compound of claim 1, wherein the compound is a compound of formula 12 or a pharmaceutically acceptable salt, hydrate, or solvate thereof:

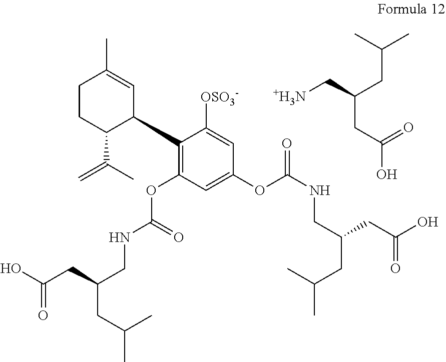

38. The compound of claim 1, wherein the compound is a compound of formula 13 or a pharmaceutically acceptable salt, hydrate, or solvate thereof:

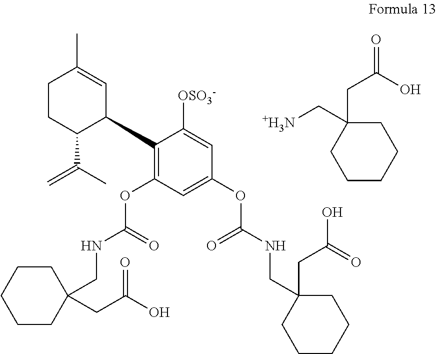

39. The compound of claim 1, wherein the compound is a compound of formula 14 or a pharmaceutically acceptable salt, hydrate, or solvate thereof:

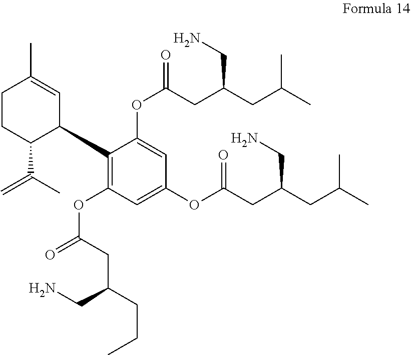 [SMILES: C=C(C)[C@@H]1CCC(C)=C[C@H]1c1c(OC(=O)C[C@H](CN)CCC)cc(OC(=O)C[C@@H](CN)CC(C)C)cc1OC(=O)C[C@@H](CN)CC(C)C]

40. The compound of claim 1, wherein the compound is a compound of formula 15 or a pharmaceutically acceptable salt, hydrate, or solvate thereof:

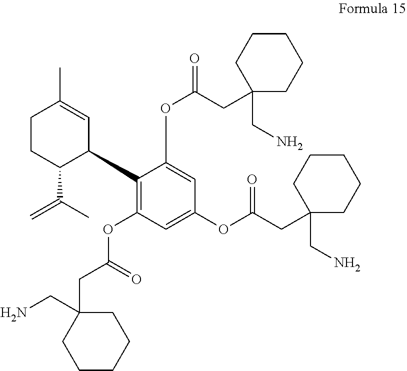 [SMILES: C=C(C)[C@@H]1CCC(C)=C[C@H]1c1c(OC(=O)CC2(CN)CCCCC2)cc(OC(=O)CC2(CN)CCCCC2)cc1OC(=O)CC1(CN)CCCCC1]

41. The compound of claim 1, wherein the compound is a compound of formula 16 or a pharmaceutically acceptable salt, hydrate, or solvate thereof:

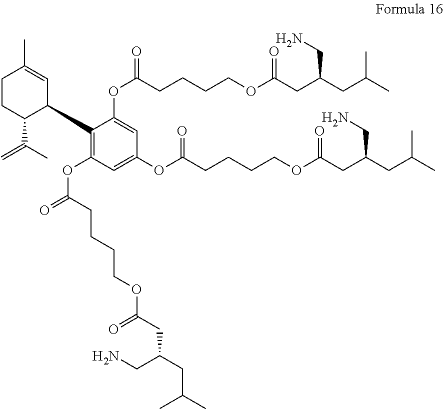 [SMILES: C=C(C)[C@@H]1CCC(C)=C[C@H]1c1c(OC(=O)CCCCOC(=O)C[C@@H](CN)CC(C)C)cc(OC(=O)CCCCOC(=O)C[C@@H](CN)CC(C)C)cc1OC(=O)CCCCOC(=O)C[C@@H](CN)CC(C)C]

42. The compound of claim 1, wherein the compound is a compound of formula 17 or a pharmaceutically acceptable salt, hydrate, or solvate thereof:

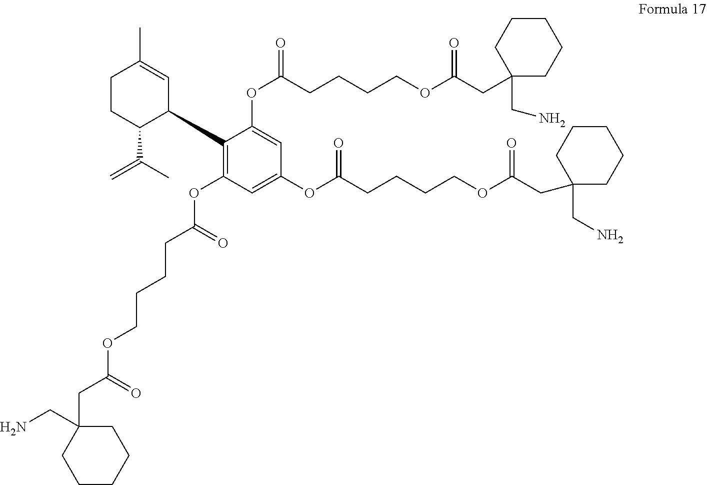 [SMILES: C=C(C)[C@@H]1CCC(C)=C[C@H]1c1c(OC(=O)CCCCOC(=O)CC2(CN)CCCCC2)cc(OC(=O)CCCCOC(=O)CC2(CN)CCCCC2)cc1OC(=O)CCCCOC(=O)CC1(CN)CCCCC1]

43. The compound of claim 1, wherein the compound is a compound of formula 18 or a pharmaceutically acceptable salt, hydrate, or solvate thereof:

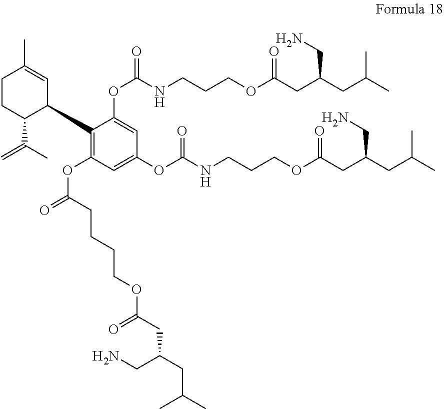 [SMILES: C=C(C)[C@@H]1CCC(C)=C[C@H]1c1c(OC(=O)CCCCOC(=O)C[C@@H](CN)CC(C)C)cc(OC(=O)NCCCOC(=O)C[C@@H](CN)CC(C)C)cc1OC(=O)NCCCOC(=O)C[C@@H](CN)CC(C)C]

44. The compound of claim 1, wherein the compound is a compound of formula 19 or a pharmaceutically acceptable salt, hydrate, or solvate thereof.

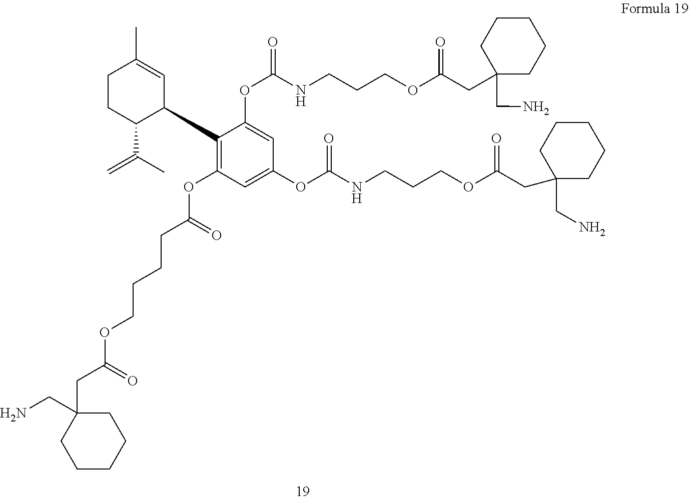 [SMILES: C=C(C)[C@@H]1CCC(C)=C[C@H]1c1c(OC(=O)CCCCOC(=O)CC2(CN)CCCCC2)cc(OC(=O)NCCCOC(=O)CC2(CN)CCCCC2)cc1OC(=O)NCCCOC(=O)CC1(CN)CCCCC1]

45. The compound of claim 1, wherein the compound is a compound of formula 20 or a pharmaceutically acceptable salt, hydrate, or solvate thereof:

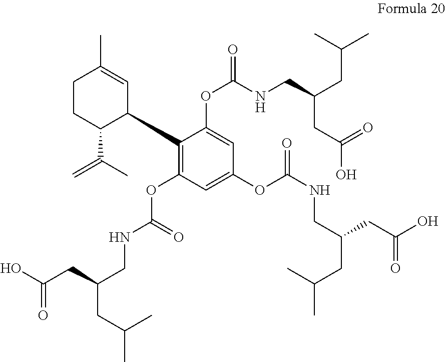 [SMILES: C=C(C)[C@@H]1CCC(C)=C[C@H]1c1c(OC(=O)NC[C@H](CC(=O)O)CC(C)C)cc(OC(=O)NC[C@H](CC(=O)O)CC(C)C)cc1OC(=O)NC[C@H](CC(=O)O)CC(C)C]

46. The compound of claim 1, wherein the compound is a compound of formula 21 or a pharmaceutically acceptable salt, hydrate, or solvate thereof:

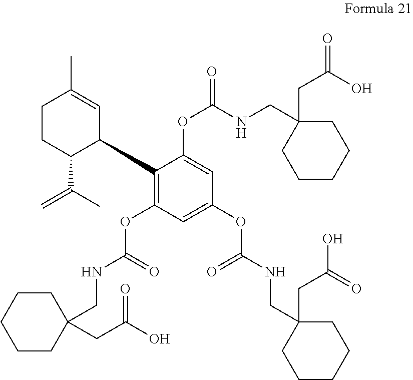 [SMILES: C=C(C)[C@@H]1CCC(C)=C[C@H]1c1c(OC(=O)NCC2(CC(=O)O)CCCCC2)cc(OC(=O)NCC2(CC(=O)O)CCCCC2)cc1OC(=O)NCC1(CC(=O)O)CCCCC1]

47. The compound of claim 1, wherein the compound is a compound of formula 22 or a pharmaceutically acceptable salt, hydrate, or solvate thereof:

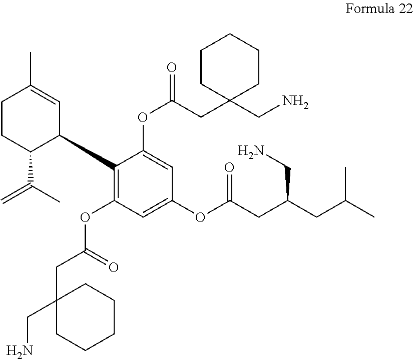 [SMILES: C=C(C)[C@@H]1CCC(C)=C[C@H]1c1c(OC(=O)CC2(CN)CCCCC2)cc(OC(=O)C[C@@H](CN)CC(C)C)cc1OC(=O)CC1(CN)CCCCC1]

48. The compound of claim 1, wherein the compound is a compound of formula 23 or a pharmaceutically acceptable salt, hydrate, or solvate thereof:

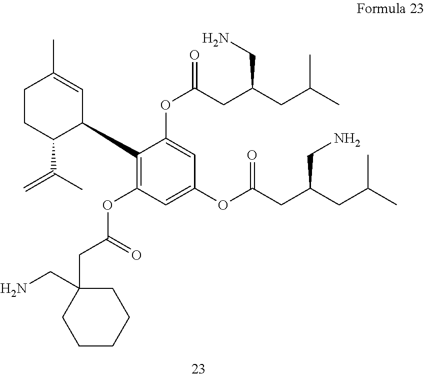 [SMILES: C=C(C)[C@@H]1CCC(C)=C[C@H]1c1c(OC(=O)C[C@@H](CN)CC(C)C)cc(OC(=O)C[C@@H](CN)CC(C)C)cc1OC(=O)CC1(CN)CCCCC1]

49. The compound of claim 1, wherein the compound is a compound of formula 24 or a pharmaceutically acceptable salt, hydrate, or solvate thereof:

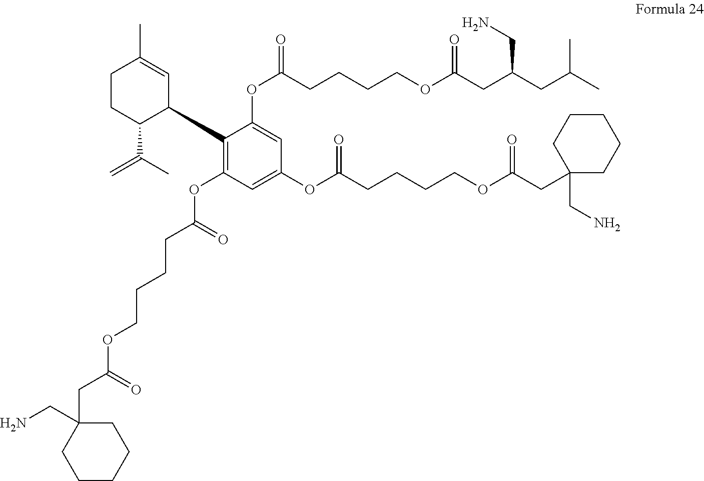 [SMILES: C=C(C)[C@@H]1CCC(C)=C[C@H]1c1c(OC(=O)CCCCOC(=O)C[C@@H](CN)CC(C)C)cc(OC(=O)CCCCOC(=O)CC2(CN)CCCCC2)cc1OC(=O)CCCCOC(=O)CC1(CN)CCCCC1]

50. The compound of claim 1, wherein the compound is a compound of formula 25 or a pharmaceutically acceptable salt, hydrate, or solvate thereof:

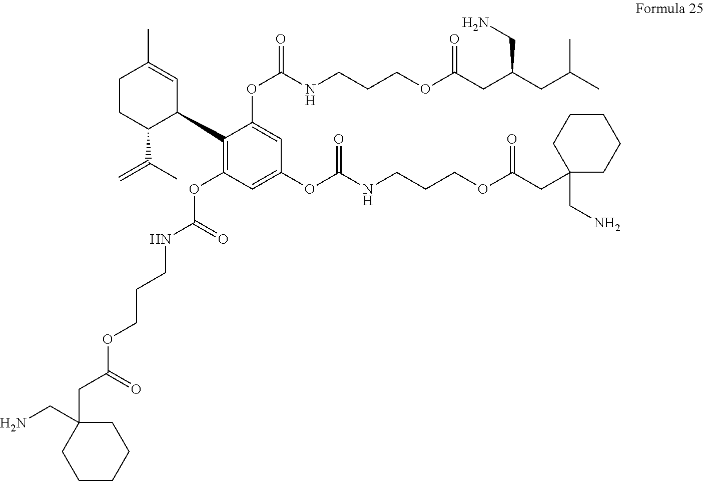 [SMILES: C=C(C)[C@@H]1CCC(C)=C[C@H]1c1c(OC(=O)NCCCOC(=O)C[C@@H](CN)CC(C)C)cc(OC(=O)NCCCOC(=O)CC2(CN)CCCCC2)cc1OC(=O)NCCCOC(=O)CC1(CN)CCCCC1]

51. The compound of claim 1, wherein the compound is a compound of formula 26 or a pharmaceutically acceptable salt, hydrate, or solvate thereof:

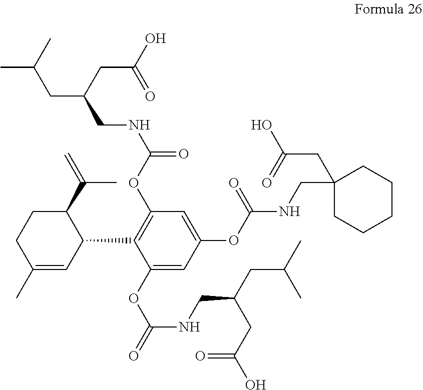 [SMILES: C=C(C)[C@@H]1CCC(C)=C[C@H]1c1c(OC(=O)NC[C@H](CC(=O)O)CC(C)C)cc(OC(=O)NCC2(CC(=O)O)CCCCC2)cc1OC(=O)NC[C@H](CC(=O)O)CC(C)C]

52. The compound of claim 1, wherein the compound is a compound of formula 27 or a pharmaceutically acceptable salt, hydrate, or solvate thereof:

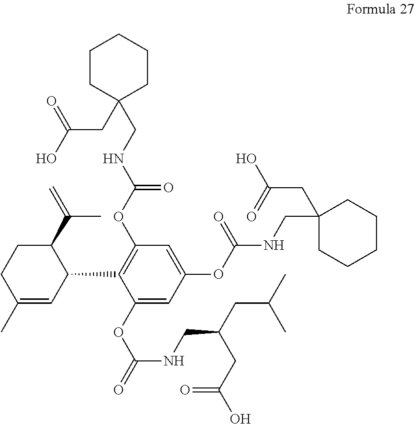 [SMILES: C=C(C)[C@@H]1CCC(C)=C[C@H]1c1c(OC(=O)NC[C@H](CC(=O)O)CC(C)C)cc(OC(=O)NCC2(CC(=O)O)CCCCC2)cc1OC(=O)NCC1(CC(=O)O)CCCCC1]

53. The compound of claim 1, wherein the compound is a compound of formula 28 or a pharmaceutically acceptable salt, hydrate, or solvate thereof:

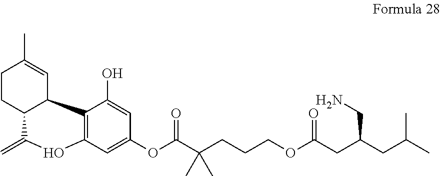 [SMILES: C=C1Oc2cc(OC(=O)C(C)(C)CCCOC(=O)C[C@@H](CN)CC(C)C)cc(O)c2[C@@H]2C=C(C)CC[C@@H]12]

54. The compound of claim 1, wherein the compound is a compound of formula 29 or a pharmaceutically acceptable salt, hydrate, or solvate thereof:

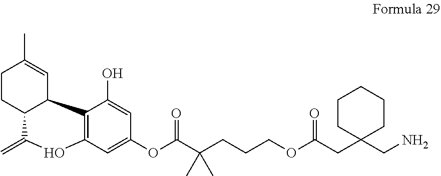 [SMILES: C=C1Oc2cc(OC(=O)C(C)(C)CCCOC(=O)CC3(CN)CCCCC3)cc(O)c2[C@@H]2C=C(C)CC[C@@H]12]

55. The compound of claim 1, wherein the compound is a compound of formula 30 or a pharmaceutically acceptable salt, hydrate, or solvate thereof:

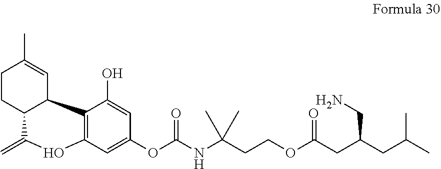 [SMILES: C=C1Oc2cc(OC(=O)NC(C)(C)CCOC(=O)C[C@@H](CN)CC(C)C)cc(O)c2[C@@H]2C=C(C)CC[C@@H]12]

56. The compound of claim 1, wherein the compound is a compound of formula 31 or a pharmaceutically acceptable salt, hydrate, or solvate thereof:

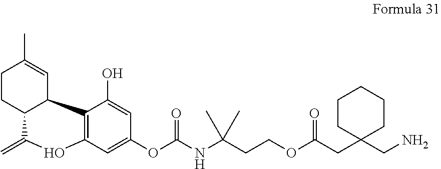 [SMILES: C=C1Oc2cc(OC(=O)NC(C)(C)CCOC(=O)CC3(CN)CCCCC3)cc(O)c2[C@@H]2C=C(C)CC[C@@H]12]

57. The compound of claim 1, wherein the compound is a compound of formula 32 or a pharmaceutically acceptable salt, hydrate, or solvate thereof:

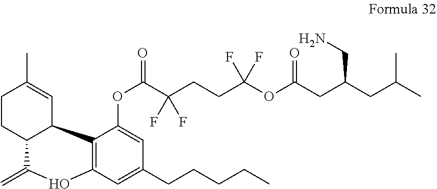 [SMILES: C=C1Oc2cc(CCCCC)cc(OC(=O)C(F)(F)CCC(F)(F)OC(=O)C[C@@H](CN)CC(C)C)c2[C@@H]2C=C(C)CC[C@@H]12]

58. The compound of claim 1, wherein the compound is a compound of formula 33 or a pharmaceutically acceptable salt, hydrate, or solvate thereof:

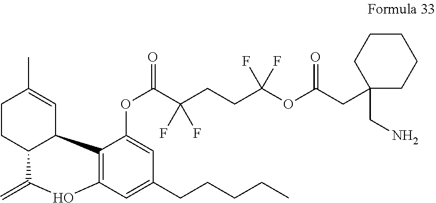 [SMILES: C=C1Oc2cc(CCCCC)cc(OC(=O)C(F)(F)CCC(F)(F)OC(=O)CC3(CN)CCCCC3)c2[C@@H]2C=C(C)CC[C@@H]12]

59. The compound of claim 1, wherein the compound is a compound of formula 34 or a pharmaceutically acceptable salt, hydrate, or solvate thereof:

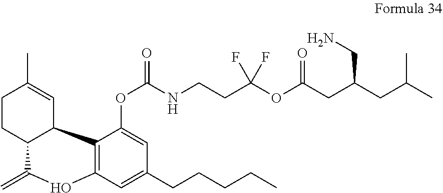 [SMILES: C=C1Oc2cc(CCCCC)cc(OC(=O)NCCC(F)(F)OC(=O)C[C@@H](CN)CC(C)C)c2[C@@H]2C=C(C)CC[C@@H]12]

60. The compound of claim 1, wherein the compound is a compound of formula 35 or a pharmaceutically acceptable salt, hydrate, or solvate thereof:

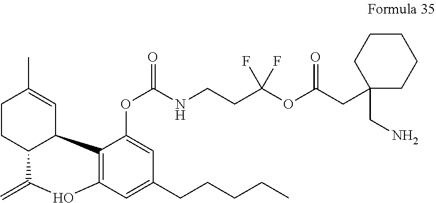 [SMILES: C=C1Oc2cc(CCCCC)cc(OC(=O)NCCC(F)(F)OC(=O)CC3(CN)CCCCC3)c2[C@@H]2C=C(C)CC[C@@H]12]

61. The compound of claim 1, wherein the compound is a compound of formula 36 or a pharmaceutically acceptable salt, hydrate, or solvate thereof:

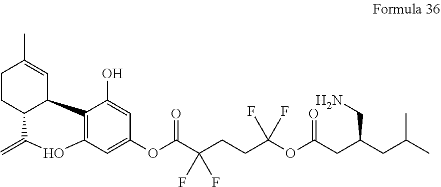 [SMILES: C=C1Oc2cc(OC(=O)C(F)(F)CCC(F)(F)OC(=O)C[C@@H](CN)CC(C)C)cc(O)c2[C@@H]2C=C(C)CC[C@@H]12]

62. The compound of claim 1, wherein the compound is a compound of formula 37 or a pharmaceutically acceptable salt, hydrate, or solvate thereof:

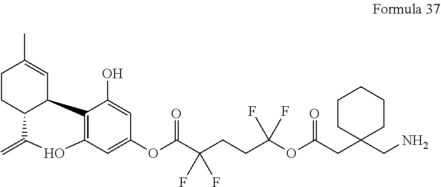 [SMILES: C=C1Oc2cc(OC(=O)C(F)(F)CCC(F)(F)OC(=O)CC3(CN)CCCCC3)cc(O)c2[C@@H]2C=C(C)CC[C@@H]12]

63. The compound of claim 1, wherein the compound is a compound of formula 38 or a pharmaceutically acceptable salt, hydrate, or solvate thereof:

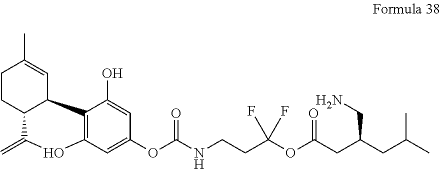 [SMILES: C=C1Oc2cc(OC(=O)NCCC(F)(F)OC(=O)C[C@@H](CN)CC(C)C)cc(O)c2[C@@H]2C=C(C)CC[C@@H]12]

64. The compound of claim 1, wherein the compound is a compound of formula 39 or a pharmaceutically acceptable salt, hydrate, or solvate thereof:

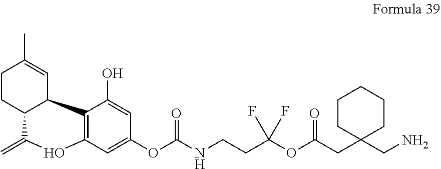 [SMILES: C=C1Oc2cc(OC(=O)NCCC(F)(F)OC(=O)CC3(CN)CCCCC3)cc(O)c2[C@@H]2C=C(C)CC[C@@H]12]

65. The use of a compound of any of claims 1 to 64 or a pharmaceutically acceptable salt, hydrate, or solvate thereof, for the treatment of one or more symptoms or conditions selected from the group consisting of: epilepsy, neuropathic pain, multiple sclerosis, seizures and postherpetic neuralgia, restless leg syndrome, trigeminal neuralgia, fibromyalgia, diabetic neuropathy, anxiety and bipolar disorders, schizophrenia, sleep disorders, post-traumatic stress disorder, anorexia, movement disorders, Tourette syndrome, glaucoma, traumatic brain injury and Crohn disease, chronic pain and spasticity, nausea and vomiting, weight gain, postherpetic neuralgia, migraine, social phobia, panic disorder, mania, and alcohol withdrawal.
